SEQUENCE LISTING (11 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 673 aa, SARS-CoV-2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 231 aa, SARS-CoV-2
SNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGL

SEQ ID NO: 4 - protein, 588 aa, SARS-CoV-2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 535 aa, Artificial sequence
  Synthetic
MACSTLPKSPKDKIDPRDLLIPLILFLSLKGARSAAPGSSPHHHHHHHHVYNITWEVTNGDRETVWAISGNHPLWTWWPVLTPDLCMLALSGPPHWGLEYQAPYSSPPGPPCCSGSSGSSAGCSRDCDEPLTSLTPRCNTAWNRLKLDQVTHKSSEGFYVCPGSHRPREAKSCGGPDSFYCASWGCETTGRVYWKPSSSWDYITVDNNLTTSQAVQVCKDNKWCNPLAIQFTNAGKQVTSWTTGHYWGLRLYVSGRDPGLTFGIRLRYQNLGPRVPIGPNPVLADQLSLPRPNPLPKPAKSPPAAASNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFN

SEQ ID NO: 6 - protein, 305 aa, murine leukemia virus MLV
MACSTLPKSPKDKIDPRDLLIPLILFLSLKGARSAAPGSSPHHHHHHHHVYNITWEVTNGDRETVWAISGNHPLWTWWPVLTPDLCMLALSGPPHWGLEYQAPYSSPPGPPCCSGSSGSSAGCSRDCDEPLTSLTPRCNTAWNRLKLDQVTHKSSEGFYVCPGSHRPREAKSCGGPDSFYCASWGCETTGRVYWKPSSSWDYITVDNNLTTSQAVQVCKDNKWCNPLAIQFTNAGKQVTSWTTGHYWGLRLYVSGRDPGLTFGIRLRYQNLGPRVPIGPNPVLADQLSLPRPNPLPKPAKSPPAA

SEQ ID NO: 7 - DNA, 1614 bp, Artificial sequence
  Synthetic
atggcgtgttcaacgctcccaaaatcccctaaagataagattgacccgcgggacctcctaatccccttaattctcttcctgtctctcaaaggggccagatccgcagcacccggctccagccctcaccatcaccaccatcatcatcacgtgtacaacattacctgggaagtgaccaatggggatcgggagacagtatgggcaatatcaggcaaccaccctctgtggacttggtggccagtcctcaccccagatttgtgtatgttagctctcagtgggccgccccactgggggctagagtatcaggccccctattcctcgcccccggggcccccttgttgctcagggagcagcgggagcagtgcaggctgttccagagactgcgacgagcccttgacctccctcacccctcggtgcaacactgcctggaacagacttaagctagaccaggtaactcataaatcaagtgagggattttatgtctgccccgggtcacatcgcccccgggaagccaagtcctgtggaggtccagactccttctactgtgcctcttggggctgcgagacaaccggtagagtatactggaagccctcctcctcttgggactacatcacagtggacaacaatctcaccactagccaggctgtccaggtatgcaaagacaataagtggtgcaatcccttggctatccagtttacaaacgccgggaaacaggtcacctcatggacaactggacactattggggtctacgtctttatgtctctgggcgggacccggggcttactttcgggatccgactcagatatcaaaatctaggacctcgggtcccgataggaccgaaccccgtcctggcagaccaactttcgctcccgcgacctaatcccctacccaaacctgccaagtctccccccgcggccgcatctaactttagagtccaaccaacagaatcgattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaactctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaattagctcgag

SEQ ID NO: 8 - DNA, 699 bp, SARS-CoV-2
gcatctaactttagagtccaaccaacagaatcgattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaactctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaattagctcgag

SEQ ID NO: 9 - DNA, 699 bp, murine leukemia virus MLV; identical to SEQ ID NO: 8

SEQ ID NO: 10 - protein, 231 aa, SARS-CoV-2
GPSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFN

SEQ ID NO: 11 - protein, 5 aa, Artificial
  Synthetic
GGGGS

(19) United States
(12) Patent Application Publication
(10) Pub. No.: US 2025/0347699 A1
(43) Pub. Date: Nov. 13, 2025

(54) SARS-COV-2 POLYPEPTIDE, ANTI-SARS-COV-2 ANTIBODIES AND USES THEREOF

(71) Applicant: Rutgers, The State University of New Jersey, New Brunswick, NJ (US)

(72) Inventors: Maria Laura Gennaro, New York, NY (US); Abraham Pinter, Brooklyn, NY (US)

(73) Assignee: Rutgers, The State University of New Jersey, New Brunswick, NJ (US)

(21) Appl. No.: 18/001,449

(22) PCT Filed: Jun. 10, 2021
(86) PCT No.: PCT/US2021/036745

§ 371 Date: Dec. 11, 2022

Related U.S. Application Data
(60) Provisional application No. 63/037,303, filed on Jun. 10, 2020.

(51) Int. Cl. G01N 33/68; C07K 14/005; G01N 33/543; G01N 33/569

(52) U.S. Cl. CPC G01N 33/6854; C07K 14/005; G01N 33/543; G01N 33/56983; C07K 2319/00; C12N 2740/13022; C12N 2770/20022; G01N 2333/165; G01N 2469/20

(57) ABSTRACT

This disclosure relates to a method of expressing the receptor-binding domain (RBD) region of the coronavirus SARS-CoV-2 Spike protein in a highly native form that is strongly reactive to natural antibodies induced upon SARS-CoV-2 infection or vaccination of humans and that more efficiently binds the angiotensin-converting enzyme 2 (ACE2) receptor. This method fuses the RBD to the C-terminus of an N-terminal fragment of the gp70 protein (the surface protein (SU) of the Friend57 strain of murine leukemia viruses). This method of expression enhances the native folding of the RBD and increases its recognition by antibodies present in immune sera and its ability to interact with the ACE2 receptor. Further disclosed are methods of using this form of RBD for various purposes.

### CROSS-REFERENCE TO RELATED APPLICATIONS

This application claims priority under 35 U.S.C. § 119(e) to U.S. Provisional Patent Application No. 63/037,303, filed Jun. 10, 2020. The foregoing application is incorporated by reference herein in its entirety.

### STATEMENT REGARDING FEDERAL FUNDING

This invention was made with government support under grant numbers TR003017 and HL 149450 awarded by the National Institutes of Health. The government has certain rights in the invention.

### REFERENCE TO A SEQUENCE LISTING

This application incorporates by reference the Sequence Listing submitted in Computer Readable Form as file Seq Listing 096747.00475.txt, created on Nov. 22, 2022 and containing 38,006 bytes.

### FIELD OF THE INVENTION

This invention relates to methods of detecting anti-SARS-CoV-2 antibodies and compositions for prophylactic or therapeutic treatment against the SARS-CoV-2.

### BACKGROUND OF THE INVENTION

SARS-CoV-2 is the virus that causes coronavirus disease 2019 (COVID-19). It contains four structural proteins, including spike(S), envelope (E), membrane (M), and nucleocapsid (N) proteins. Among them, the spike protein plays the most important roles in viral attachment, fusion, and entry, and it serves as a target for development of antibodies, entry inhibitors, and vaccines. The spike protein mediates viral entry into host cells by first binding to a host receptor through the receptor-binding domain (RBD) in the S1 subunit and then fusing the viral and host membranes through the S2 subunit. SARS-CoV-2 and MERS-COV RBDs recognize different receptors. SARS-CoV-2 recognizes angiotensin-converting enzyme 2 (ACE2) as its receptor, whereas MERS-COV recognizes dipeptidyl peptidase 4 (DPP4) as its receptor.

The numbers of the COVID-19 cases are staggering. As of Jun. 3, 2021, a total of 171 million confirmed cases of COVID-19 were reported, including 3,686,142 deaths, in the United States and at least 85 other countries and/or territories. Among those testing positive to qPCR assays for viral nucleic acid in oropharyngeal swabs, many experienced only mild symptoms, while others developed moderate to severe disease, which may progress to acute respiratory distress syndrome and death. For example, in a report from China, among the hospitalized patients, ˜15% were admitted as severe disease; of these, more than one-third required mechanical ventilation. Thus, a spectrum of disease severity exists in people infected with SARS-CoV-2. Patient characteristics such as age (>60 years) and comorbidities (e.g., hypertension and diabetes) are most likely associated with COVID-19 severity. However, these variables are insufficient to explain the clinical spectrum of COVID-19. The factors underlying disease severity remain largely unknown.

There is a pressing need for diagnostic and screening methods to identify the presence of the virus and measure the level of protective immunity in, e.g., mammalian tissue or serum, as well as compositions for prophylactic or therapeutic treatment against the SARS-CoV-2.

### SUMMARY OF THE INVENTION

This disclosure addresses the need mentioned above in a number of aspects. In one aspect, this disclosure provides a method of detecting an antibody or antigen-binding protein that specifically binds to a SARS-CoV-2 antigen in a sample from a subject. The method comprises: (i) contacting the sample with a SARS-CoV-2 antigen, under conditions suitable for binding the antibody or antigen-binding protein to the SARS-CoV-2 antigen; and (ii) detecting the binding of the antibody or antigen-binding protein to the SARS-CoV-2 antigen. In some embodiments, the SARS-CoV-2 is a human or an animal SARS-CoV-2.

In some embodiments, the SARS-CoV-2 antigen is immobilized on a solid phase substrate either directly or through binding to an immobilized (capture) antibody, and the method further comprises: (a) contacting the sample with the solid phase substrate under conditions suitable for binding the antibody or antigen-binding protein to the SARS-CoV-2 antigen, and (b) detecting binding of the antibody or antigen-binding protein to the solid phase substrate. The binding of the antibody or antigen-binding protein to the solid phase substrate is indicative of the subject having the antibody or antigen-binding protein that specifically binds the SARS-CoV-2 antigen.

In some embodiments, the method further comprises identifying the antibody or antigen-binding protein after the antibody or antigen-binding protein is detected.

In another aspect, this disclosure provides a method of isolating anti-SARS-CoV-2 antibodies. The method comprises: (i) contacting a sample from a subject infected with or vaccinated against a SARS-CoV-2 with SARS-CoV-2 antigen that is immobilized on a surface of a solid phase substrate; (ii) allowing SARS-CoV-2 antibodies in the sample to bind to the SARS-CoV-2 antigen; (iii) optionally washing the solid phase substrate; (iv) releasing bound SARS-CoV-2 antibodies from the solid phase substrate; and (v) collecting the SARS-CoV-2 antibodies released from the solid phase substrate. In some embodiments, the method further comprises identifying the antibody or antigen-binding protein after the antibodies are isolated.

In some embodiments, the SARS-CoV-2 antigen comprises a spike protein of a SARS-CoV-2 or fragment/variant thereof, a nucleocapsid protein or fragment/variant thereof, or a combination thereof. In some embodiments, the SARS-CoV-2 antigen comprises a S1 subunit or a S2 subunit of the spike protein of the SARS-CoV-2, or a fragment/variant thereof. In some embodiments, the SARS-CoV-2 antigen comprises a receptor-binding domain (RBD) in the spike protein of the SARS-CoV-2, or fragment/variant thereof.

In some embodiments, the SARS-CoV-2 antigen comprises an amino acid sequence of SEQ ID NO: 5 or an amino acid sequence having at least 75% (e.g., 75%, 80%, 85%, 90%, 95%, 96%, 97%, 98%, 99%) sequence identity to SEQ ID NO: 5.

In some embodiments, the SARS-CoV-2 antigen comprises a fusion polypeptide comprising the spike protein of the SARS-CoV-2 or fragment/variant thereof (e.g., RBD) fused to a gp70 polypeptide or fragment/variant thereof. The spike protein or fragment/variant thereof (e.g., RBD) is fused to the C-terminus of the gp70 polypeptide or fragment/variant thereof. In some embodiments, the spike protein or fragment/variant thereof is fused to the C-terminus of the gp70 polypeptide or fragment/variant thereof via a peptide linker. In some embodiments, the spike protein or fragment/variant thereof comprises the RBD. In some embodiments, the RBD comprises the amino acids 316-542 of the spike protein.

In some embodiments, the fusion polypeptide comprises an amino acid sequence of SEQ ID NO: 5 or an amino acid sequence having at least 75% (e.g., 75%, 80%, 85%, 90%, 95%, 96%, 97%, 98%, 99%) sequence identity to SEQ ID NO: 5.

In some embodiments, the SARS-CoV-2 antigen comprises a mixture of two or more of the RBD, the S1 subunit, the S2 subunit, the spike protein, the nucleocapsid protein. In some embodiments, the SARS-CoV-2 antigen comprises the fusion polypeptide (e.g., gp70-RBD) and the S2 subunit.

In some embodiments, the sample comprises a saliva, blood, serum, or plasma sample. In some embodiments, the sample may be obtained from a subject that is asymptomatic. In some embodiments, the subject either is having an active infection or has been exposed to the SARS-CoV-2. In some embodiments, the subject has been treated with an anti-inflammatory agent or an antiviral agent or therapy.

In some embodiments, the antiviral agent or therapy comprises a convalescent plasma therapy. In some embodiments, the antiviral agent may include: a nucleoside analog, a peptoid, an oligopeptide, a polypeptide, a protease inhibitor, a 3C-like protease inhibitor, a papain-like protease inhibitor, an anti-IL6 inhibitor, and an inhibitor of an RNA dependent RNA polymerase. In some embodiments, the antiviral agent may include acyclovir, gancyclovir, vidarabine, foscarnet, cidofovir, amantadine, ribavirin, trifluorothymidine, zidovudine, didanosine, zalcitabine, tocilizumab, an interferon, and a combination thereof.

In some embodiments, the solid phase substrate may include capture antibodies, microparticles, microbeads, magnetic beads, membrane, and an affinity purification column.

In some embodiments, the methods described above may include detecting fluorescence or chemiluminescence. In some embodiments, the method may include a competitive binding assay or an immunoassay (e.g., ELISA).

In some embodiments, the competitive binding assay may include detecting the binding of the antibody to the SARS-CoV-2 antigen in the presence of an ACE2 polypeptide or fragment thereof. The ACE2 polypeptide or fragment thereof is capable of binding to the SARS-CoV-2 antigen.

In some embodiments, the step of detecting comprises detecting the antibody or antigen-binding protein bound to the SARS-CoV-2 antigen or the solid phase substrate using a second SARS-CoV-2 antigen that interacts with the antibody or antigen-binding protein. In some embodiments, the second SARS-CoV-2 antigen comprises a detection agent. In some embodiments, the detection agent comprises a biotin moiety. In some embodiments, the second SARS-CoV-2 antigen is biotinylated.

In some embodiments, the methods described above may include contacting one or more secondary antibodies with the sample. The secondary antibodies are capable of binding to an anti-SARS-CoV-2 antibody (e.g., with an IgG, IgM, or IgA heavy chain or kappa and/or lambda light chain) or antigen-binding protein. In some embodiments, the secondary antibodies (for detecting, e.g., IgG, IgM, IgA heavy chain) may be conjugated to a label, e.g., a fluorescent label, a chemiluminescent label, a radiolabel, and an enzyme.

In yet another aspect, this disclosure also provides a polypeptide comprising a spike polypeptide of a SARS-CoV-2 fused to a gp70 polypeptide. In some embodiments, the spike polypeptide may be fused to the N- or C-terminus of the gp70 polypeptide. In some embodiments, the polypeptide comprises an amino acid sequence of SEQ ID NO: 5 or an amino acid sequence having at least 75% sequence identity to SEQ ID NO: 5.

Also provided in this disclosure are (a) a polynucleotide comprising a polynucleotide sequence that encodes the polypeptide described above; (b) a vector comprising the polynucleotide as described; (c) a host cell comprising the vector as described; and (d) a mammalian cell line expressing the polypeptide as described.

In some embodiments, the polynucleotide comprises the polynucleotide sequence of SEQ ID NOs: 7 or a polynucleotide sequence having at least 75% sequence identity to the polynucleotide sequence of SEQ ID NOs: 7.

In another aspect, this disclosure further provides an immunogenic composition comprising the polypeptide or the polynucleotide, as described above, and optionally a carrier. Also provided is a pharmaceutical composition comprising an effective amount of the polypeptide described above and optionally a carrier or excipient. In some embodiments, the effective amount of the polypeptide is effective for inhibition of binding of SARS-CoV-2 with the ACE2 receptor on the surface of target cells, or with fusion with, or entry into, mammalian cells or for treatment of SARS-CoV-2 infection. In some embodiments, the carrier is an adjuvant, including: aluminum hydroxide, lipid A, killed bacteria, polysaccharide, mineral oil, Freund's incomplete adjuvant, Freund's complete adjuvant, aluminum phosphate, iron, zinc, a calcium salt, acylated tyrosine, an acylated sugar, a cationically derivatized polysaccharide, an anionically derivatized polysaccharide, a polyphosphazene, a biodegradable microsphere, a monophosphoryl lipid A, and quil A.

In another aspect, this disclosure further provides an isolated antibody or antigen-binding fragment thereof that specifically binds the polypeptide described above. In some embodiments, the isolated antibody or antigen-binding fragment thereof is isolated from the sample of the subject that has been administered the polypeptide, the polynucleotide, or immunogenic composition, or a combination thereof, as described above, or from a sample of a subject recovered from a SARS-CoV-2 infection.

In some embodiments, the antibody can be a monoclonal antibody, a polyclonal antibody, a single-chain antibody, an antigen-binding antibody fragment, or a humanized antibody. In some embodiments, the antibody or antigen-binding fragment thereof may be coupled to a detectable tag. In some embodiments, the detectable tag can be a fluorescent protein, a fluorescent marker, a radiolabel, an enzyme, or an affinity tag.

In another aspect, this disclosure additionally provides a method of treating or inhibiting a SARS-CoV-2 infection. The method includes administering to a mammal in need a therapeutically effective amount of the polypeptide, the polynucleotide, the pharmaceutical composition, or the isolated antibody or antigen-binding fragment thereof, as described above.

In another aspect, this disclosure further provides a method for eliciting a detectable immune response to the spike polypeptide or fragment thereof. Also provided is a method of vaccinating a subject. These methods comprise administering to a mammal the polypeptide described above.

Also within the scope of this disclosure is a kit comprising an effective amount of the polypeptide, the polynucleotide, the pharmaceutical compositions, or the isolated antibody or antigen-binding fragment thereof, as described above.

In another aspect, this disclosure also provides a kit for detection or diagnosis of a SARS-CoV-2 infection or determining a level of protective immunity. In some embodiments, the kit comprises a first detection reagent comprising the polypeptide as described above. In some embodiments, the polypeptide comprises a spike polypeptide fused to a gp70 polypeptide. In some embodiments, the polypeptide comprises an amino acid sequence of SEQ ID NO: 5 or an amino acid sequence having at least 75% sequence identity to SEQ ID NO: 5. In some embodiments, the kit further comprises a second detection reagent comprising the polypeptide described above. In some embodiments, the polypeptide contained in the second detection reagent comprises a detection label. In some embodiments, the detection label comprises a biotin moiety. In some embodiments, the polypeptide is biotinylated.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a set of graphs showing the sensitivity of the two RBD forms as antigens that were tested for the detection and titration of antibodies in SARS-CoV-2 convalescent patient sera. The sera used in this assay included one high titer serum (LK #74) and two low titer sera (CH-1944 and CH-1945) along with one normal human serum control. The results showed a clear advantage of the gp70-RBD antigen over the RBD-BEI antigen for all three positive sera.

FIGS. 2A and 2B are a set of graphs showing characterization of the interaction between the RBD antigen and ACE2. Two procedures were examined to measure RBD/sol-ACE2 binding.

FIG. 3 is a diagram showing an example method using labeled antigen to detect free arm of captured antibodies detects only specific antibodies, but not non-specifically captured.

FIGS. 4A, 4B, 4C, and 4D are a set of graphs showing binding of sera from SARS-CoV-2-infected adolescents to our gp70-RBD antigen (diamonds) or to the gp70-carrier domain (squares). FIGS. 4A and 4B show results from an adolescent cohort infected with SARS-CoV-2 who experienced mild symptoms (sera are sorted by level of reactivity against the RBD antigen attached to the plate). FIG. 4A shows curves obtained with RBD-b detection reagent. FIG. 4B shows curves obtained with a standard secondary reagent. FIGS. 4C and 4D show a similar analysis for a pre-Covid panel of sera. For both sample sets, the standard detection method gave large backgrounds, while the antigen detection reagent gave no non-specific reactivity.

FIG. 5 shows correlations for psV neutralization titers (diamonds) vs. binding activity measured either with biotinylated RBD (squares) or with standard secondary antibody detection reagent (triangles). Correlation coefficients are shown below the plot for IC50s and different binding formats. Results are shown for plasma tested at a 1:2 dilution.

FIG. 6 is a structural representation showing the RBD in light grey and the ACE-2 receptor in dark grey. Converting the three highlighted Asn residues (e.g., N360, N394, and N388) to N-linked glycosylation sites by modifying the N+2 residue to Ser or Thr have do not to effect ACE-2 binding or RBD expression.

### DETAILED DESCRIPTION OF THE INVENTION

This disclosure provides a novel method for detecting anti-SARS-CoV-2 antibodies/immunity in an individual. The method may include a serological assay using an ELISA-based method to detect the presence or absence of antibodies against the specific SARS-CoV-2 antigens (e.g., RBD, gp70-RBD, S1 subunit, S2 subunit, nucleocapsid, a fragment/variant thereof or a combination thereof). The presence of anti-SARS-CoV-2 antibodies against a SARS-CoV-2 specific antigen in a biological sample of an individual indicates that the individual either is having an active infection or has been exposed to the SARS-CoV-2. The disclosed method can be implemented in a high-throughput format to study the extent of past/present infection in large populations. This disclosure further provides compositions comprising novel SARS-CoV-2 antigens, and/or antibodies developed therefrom, for prophylactic or therapeutic treatment against SARS-CoV-2 infections.

### A. Methods of Detecting Antibodies or Antigen-Binding Proteins Against SARS-COV-2 Antigens

In one aspect, this disclosure provides a method of detecting an antibody or antigen-binding protein that specifically binds to a SARS-CoV-2 antigen in a sample from a subject. The method comprises: (i) contacting the sample with a SARS-CoV-2 antigen, under conditions suitable for binding the antibody or antigen-binding protein to the SARS-CoV-2 antigen; and (ii) detecting the binding of the antibody or antigen-binding protein to the SARS-CoV-2 antigen. In some embodiments, the SARS-CoV-2 is a human or an animal SARS-CoV-2.

In some embodiments, the SARS-CoV-2 antigen is immobilized on a solid phase substrate either directly or through binding to an immobilized (capture) antibody. The method further comprises: (a) contacting the sample with the solid phase substrate under conditions suitable for binding the antibody or antigen-binding protein to the SARS-CoV-2 antigen, and (b) detecting binding of the antibody or antigen-binding protein to the solid phase substrate. The binding of the antibody or antigen-binding protein to the solid phase substrate is indicative of the subject having the antibody or antigen-binding protein that specifically binds the SARS-CoV-2 antigen.

In some embodiments, the above methods further comprise identifying the antibody or antigen-binding protein after the antibody or antigen-binding protein is detected.

In some embodiments, the step of detecting comprises detecting the antibody or antigen-binding protein bound to the SARS-CoV-2 antigen or the solid phase substrate using a second SARS-CoV-2 antigen that interacts with the antibody or antigen-binding protein. In some embodiments, the second SARS-CoV-2 antigen comprises a detection agent. In some embodiments, the detection agent comprises a biotin moiety. In some embodiments, the second SARS-CoV-2 antigen is biotinylated.

a. SARS-CoV-2 Antigens

In some embodiments, the SARS-CoV-2 antigen comprises a spike protein of a SARS-CoV-2 (e.g., RBD, S1 subunit, S2 subunit) or fragment/variant thereof, used in combination with a nucleocapsid protein or fragment/variant thereof or some other viral antigen. For example, the SARS-CoV-2 antigen may include a S1 subunit or a S2 subunit of the spike protein of the SARS-CoV-2 or fragment/variant thereof. In some embodiments, the SARS-CoV-2 antigen comprises a RBD in the S1 subunit of the spike protein or fragment/variant thereof.

The spike protein is important because it is present on the outside of intact SARS-CoV-2, and among the SARS-CoV-2 structural proteins, it plays the most important roles in viral attachment, fusion, and entry. Representative sequences of the spike protein and subunits/sub-domains/fusion proteins thereof are provided in Table 1 below.

TABLE 1
Representative Sequences

| SEQ | | |
|---|---|---|
| ID | | |
| NO | SEQUENCE | NOTES |
| 1 | MFVFLVLLPLVSSQCVNLTTRTQLPPAYTNSFTRGVYYPDKVFRS | NCBI |
| | SVLHSTQDLFLPFFSNVTWFHAIHVSGTNGTKRFDNPVLPFNDGV | Accession ID: |
| | YFASTEKSNIIRGWIFGTTLDSKTQSLLIVNNATNVVIKVCEFQF | NC_045512.2 |
| | CNDPFLGVYYHKNNKSWMESEFRVYSSANNCTFEYVSQPFLMDLE | |
| | GKQGNFKNLREFVFKNIDGYFKIYSKHTPINLVRDLPQGFSALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVRFPNITNLCPFGEVFNATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKFLPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPRRARSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLFNKVTLADAGFIKQYGDCLGDIAARDLICAQKF | |
| | NGLTVLPPLLTDEMIAQYTSALLAGTITSGWTFGAGAALQIPFAM | |
| | QMAYRFNGIGVTQNVLYENQKLIANQFNSAIGKIQDSLSSTASAL | |
| | GKLQDVVNQNAQALNTLVKQLSSNFGAISSVLNDILSRLDKVEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPWY | |
| | IWLGFIAGLIAIVMVTIMLCCMTSCCSCLKGCCSCGSCCKFDEDD | |
| | SEPVLKGVKLHYT | |
| 2 | SQCVNLTTRTQLPPAYTNSFTRGVYYPDKVFRSSVLHSTQDLFLP | S1 subunit |
| | FFSNVTWFHAIHVSGTNGTKRFDNPVLPFNDGVYFASTEKSNIIR | (residues 13- |
| | GWIFGTTLDSKTQSLLIVNNATNVVIKVCEFQFCNDPFLGVYYHK | 685) |
| | NNKSWMESEFRVYSSANNCTFEYVSQPFLMDLEGKQGNFKNLREF | |
| | VFKNIDGYFKIYSKHTPINLVRDLPQGFSALEPLVDLPIGINITR | |
| | FQTLLALHRSYLTPGDSSSGWTAGAAAYYVGYLQPRTFLLKYNEN | |
| | GTITDAVDCALDPLSETKCTLKSFTVEKGIYQTSNFRVQPTESIV | |
| | RFPNITNLCPFGEVENATRFASVYAWNRKRISNCVADYSVLYNSA | |
| | SFSTFKCYGVSPTKLNDLCFTNVYADSFVIRGDEVRQIAPGQTGK | |
| | IADYNYKLPDDFTGCVIAWNSNNLDSKVGGNYNYLYRLFRKSNLK | |
| | PFERDISTEIYQAGSTPCNGVEGENCYFPLQSYGFQPTNGVGYQP | |
| | YRVVVLSFELLHAPATVCGPKKSTNLVKNKCVNFNFNGLTGTGVL | |
| | TESNKKFLPFQQFGRDIADTTDAVRDPQTLEILDITPCSFGGVSV | |
| | ITPGTNTSNQVAVLYQDVNCTEVPVAIHADQLTPTWRVYSTGSNV | |
| | FQTRAGCLIGAEHVNNSYECDIPIGAGICASYQTQTNSPRRAR | |
| 3 | SNFRVQPTESIVRFPNITNLCPFGEVFNATRFASVYAWNRKRISN | RBD |
| | CVADYSVLYNSASFSTFKCYGVSPTKLNDLCFTNVYADSFVIRGD | (residues 316- |
| | EVRQIAPGQTGKIADYNYKLPDDFTGCVIAWNSNNLDSKVGGNYN | 542) |
| | YLYRLFRKSNLKPFERDISTEIYQAGSTPCNGVEGFNCYFPLQSY | |
| | GFQPTNGVGYQPYRVVVLSFELLHAPATVCGPKKSTNLVKNKCVN | |
| | FNFNGL | |
| 4 | SVASQSIIAYTMSLGAENSVAYSNNSIAIPTNFTISVTTEILPVS | S2 subunit |
| | MTKTSVDCTMYICGDSTECSNLLLQYGSFCTQLNRALTGIAVEQD | (residues 686- |
| | KNTQEVFAQVKQIYKTPPIKDFGGFNFSQILPDPSKPSKRSFIED | 1273 |
| | LLFNKVTLADAGFIKQYGDCLGDIAARDLICAQKENGLTVLPPLL | |
| | TDEMIAQYTSALLAGTITSGWTFGAGAALQIPFAMQMAYRFNGIG | |
| | VTQNVLYENQKLIANQFNSAIGKIQDSLSSTASALGKLQDVVNQN | |
| | AQALNTLVKQLSSNFGAISSVLNDILSRLDKVEAEVQIDRLITGR | |
| | LQSLQTYVTQQLIRAAEIRASANLAATKMSECVLGQSKRVDFCGK | |
| | GYHLMSFPQSAPHGVVFLHVTYVPAQEKNFTTAPAICHDGKAHFP | |
| | REGVFVSNGTHWFVTQRNFYEPQIITTDNTFVSGNCDVVIGIVNN | |
| | TVYDPLQPELDSFKEELDKYFKNHTSPDVDLGDISGINASVVNIQ | |
| | KEIDRLNEVAKNLNESLIDLQELGKYEQYIKWPWYIWLGFIAGLI | |
| | AIVMVTIMLCCMTSCCSCLKGCCSCGSCCKFDEDDSEPVLKGVKL | |
| | HYT | |
| 5 | MACSTLPKSPKDKIDPRDLLIPLILFLSLKGARSAAPGSSPHHHH | gp70-RBD |
| | HHHHVYNITWEVTNGDRETVWAISGNHPLWTWWPVLTPDLCMLAL | |
| | SGPPHWGLEYQAPYSSPPGPPCCSGSSGSSAGCSRDCDEPLTSLT | |
| | PRCNTAWNRLKLDQVTHKSSEGFYVCPGSHRPREAKSCGGPDSFY | |
| | CASWGCETTGRVYWKPSSSWDYITVDNNLTTSQAVQVCKDNKWCN | |
| | PLAIQFTNAGKQVTSWTTGHYWGLRLYVSGRDPGLTFGIRLRYQN | |
| | LGPRVPIGPNPVLADQLSLPRPNPLPKPAKSPPAAASNFRVQPTE | |
| | SIVRFPNITNLCPFGEVFNATRFASVYAWNRKRISNCVADYSVLY | |
| | NSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKS | |
| | NLKPFERDISTEIYQAGSTPCNGVEGFNCYFPLQSYGFQPTNGVG | |
| | YQPYRVVVLSFELLHAPATVCGPKKSTNLVKNKCVNFNFN | |
| 6 | MACSTLPKSPKDKIDPRDLLIPLILFLSLKGARSAAPGSSPHHHH | gp70 |
| | HHHHVYNITWEVTNGDRETVWAISGNHPLWTWWPVLTPDLCMLAL | |
| | SGPPHWGLEYQAPYSSPPGPPCCSGSSGSSAGCSRDCDEPLTSLT | |
| | PRCNTAWNRLKLDQVTHKSSEGFYVCPGSHRPREAKSCGGPDSFY | |
| | CASWGCETTGRVYWKPSSSWDYITVDNNLTTSQAVQVCKDNKWCN | |
| | PLAIQFTNAGKQVTSWTTGHYWGLRLYVSGRDPGLTFGIRLRYQN | |
| | LGPRVPIGPNPVLADQLSLPRPNPLPKPAKSPPAA | |
| 7 | ATGGCGTGTTCAACGCTCCCAAAATCCCCTAAAGATAAGATTGAC | gp70-RBD |
| | CCGCGGGACCTCCTAATCCCCTTAATTCTCTTCCTGTCTCTCAAA | |
| | GGGGCCAGATCCGCAGCACCCGGCTCCAGCCCTCACCATCACCAC | |
| | CATCAtcatcacGTgTACAACATTACCTGGGAAGTGACCAATGGG | |
| | GATCGGGAGACAGTATGGGCAATATCAGGCAACCACCCTCTGTGG | |
| | ACTTGGTGGCCAGTCCTCACCCCAGATTTGTGTATGTTAGCTCTC | |
| | AGTGGGCCGCCCCACTGGGGGCTAGAGTATCAGGCCCCCTATTCC | |
| | TCGCCCCCGGGGCCCCCTTGTTGCTCAGGGAGCAGCGGGAGCAGT | |
| | GCAGGCTGTTCCAGAGACTGCGACGAGCCCTTGACCTCCCTCACC | |
| | CCTCGGTGCAACACTGCCTGGAACAGACTTAAGCTAGACCAGGTA | |
| | ACTCATAAATCAAGTGAGGGATTTTATGTCTGCCCCGGGTCACAT | |
| | CGCCCCCGGGAAGCCAAGTCCTGTGGAGGTCCAGACTCCTTCTAC | |
| | TGTGCCTCTTGGGGCTGCGAGACAACCGGTAGAGTATACTGGAAG | |
| | CCCTCCTCCTCTTGGGACTACATCACAGTGGACAACAATCTCACC | |
| | ACTAGCCAGGCTGTCCAGGTATGCAAAGACAATAAGTGGTGCAAT | |
| | CCCTTGGCTATCCAGTTTACAAACGCCGGGAAACAGGTCACCTCA | |
| | TGGACAACTGGACACTATTGGGGTCTACGTCTTTATGTCTCTGGG | |
| | CGGGACCCGGGGCTTACTTTCGGGATCCGACTCAGATATCAAAAT | |
| | CTAGGACCTCGGGTCCCGATAGGACCGAACCCCGTCCTGGCAGAC | |
| | CAACTTTCGCTCCCGCGACCTAATCCCCTACCCAAACCTGCCAAG | |
| | TCTCCCCCCgcggccgcatctaactttagagtccaaccaacagaa | |
| | tcgattgttagatttcctaatattacaaacttgtgcccttttggt | |
| | gaagtttttaacgccaccagatttgcatctgtttatgcttggaac | |
| | aggaagagaatcagcaactgtgttgctgattattctgtcctatat | |
| | aattccgcatcattttccacttttaagtgttatggagtgtctcct | |
| | actaaattaaatgatctctgctttactaatgtctatgcagattca | |
| | tttgtaattagaggtgatgaagtcagacaaatcgctccagggcaa | |
| | actggaaagattgctgattataattataaattaccagatgatttt | |
| | acaggctgcgttatagcttggaactctaacaatcttgattctaag | |
| | gttggtggtaattataattacctgtatagattgtttaggaagtct | |
| | aatctcaaaccttttgagagagatatttcaactgaaatctatcag | |
| | gccggtagcacaccttgtaatggtgttgaaggttttaattgttac | |
| | tttcctttacaatcatatggtttccaacccactaatggtgttggt | |
| | taccaaccatacagagtagtagtactttcttttgaacttctacat | |
| | gcaccagcaactgtttgtggacctaaaaagtctactaatttggtt | |
| | aaaaacaaatgtgtcaatttcaacttcaattagctcgag | |
| 8 | gcatctaactttagagtccaaccaacagaatcgattgttagattt | RBD |
| | cctaatattacaaacttgtgcccttttggtgaagtttttaacgcc | |
| | accagatttgcatctgtttatgcttggaacaggaagagaatcagc | |
| | aactgtgttgctgattattctgtcctatataattccgcatcattt | |
| | tccacttttaagtgttatggagtgtctcctactaaattaaatgat | |
| | ctctgctttactaatgtctatgcagattcatttgtaattagaggt | |
| | gatgaagtcagacaaatcgctccagggcaaactggaaagattgct | |
| | gattataattataaattaccagatgattttacaggctgcgttata | |
| | gcttggaactctaacaatcttgattctaaggttggtggtaattat | |
| | aattacctgtatagattgtttaggaagtctaatctcaaacctttt | |
| | gagagagatatttcaactgaaatctatcaggccggtagcacacct | |
| | tgtaatggtgttgaaggttttaattgttactttcctttacaatca | |
| | tatggtttccaacccactaatggtgttggttaccaaccatacaga | |
| | gtagtagtactttcttttgaacttctacatgcaccagcaactgtt | |
| | tgtggacctaaaaagtctactaatttggttaaaaacaaatgtgtc | |
| | aatttcaacttcaattagctcgag | |
| 9 | gcatctaactttagagtccaaccaacagaatcgattgttagattt | gp70 |
| | cctaatattacaaacttgtgcccttttggtgaagtttttaacgcc | |
| | accagatttgcatctgtttatgcttggaacaggaagagaatcagc | |
| | aactgtgttgctgattattctgtcctatataattccgcatcattt | |
| | tccacttttaagtgttatggagtgtctcctactaaattaaatgat | |
| | ctctgctttactaatgtctatgcagattcatttgtaattagaggt | |
| | gatgaagtcagacaaatcgctccagggcaaactggaaagattgct | |
| | gattataattataaattaccagatgattttacaggctgcgttata | |
| | gcttggaactctaacaatcttgattctaaggttggtggtaattat | |
| | aattacctgtatagattgtttaggaagtctaatctcaaacctttt | |
| | gagagagatatttcaactgaaatctatcaggccggtagcacacct | |
| | tgtaatggtgttgaaggttttaattgttactttcctttacaatca | |
| | tatggtttccaacccactaatggtgttggttaccaaccatacaga | |
| | gtagtagtactttcttttgaacttctacatgcaccagcaactgtt | |
| | tgtggacctaaaaagtctactaatttggttaaaaacaaatgtgtc | |
| | aatttcaacttcaattagctcgag | |
| 10 | GPSNFRVQPTESIVRFPNITNLCPFGEVENATRFASVYAWNRKRI | Cleaved RBD |
| | SNCVADYSVLYNSASFSTFKCYGVSPTKLNDLCFTNVYADSFVIR | 316-542 |
| | GDEVRQIAPGQTGKIADYNYKLPDDFTGCVIAWNSNNLDSKVGGN | |
| | YNYLYRLFRKSNLKPFERDISTEIYQAGSTPCNGVEGFNCYFPLQ | |
| | SYGFQPTNGVGYQPYRVVVLSFELLHAPATVCGPKKSTNLVKNKC | |
| | VNFNFN | |

In some embodiments, the SARS-CoV-2 antigen comprises an amino acid sequence of SEQ ID NOs: 1-4 or an amino acid sequence having at least 75% (e.g., 75%, 80%, 85%, 90%, 95%, 96%, 97%, 98%, 99%) sequence identity to SEQ ID NOs: 1-4.

In some embodiments, the SARS-CoV-2 antigen may include a fusion polypeptide comprising the spike protein of the SARS-CoV-2 or fragment/variant thereof (e.g., RBD) fused to the Murine Leukemia Virus (MuLV) Surface (SU) protein gp70 polypeptide or fragment/variant thereof. The gp70 domain acts as a large protein tag that facilitates the purification and detection of the attached RBD domain and may also assist in the correct folding and glycosylation of the/rbd domain. The spike protein or fragment/variant thereof (e.g., RBD) is fused to the N- or C-terminus of the gp70 polypeptide or fragment/variant thereof. In one example, the spike protein or fragment/variant thereof can be fused to the N- or C-terminus of the gp70 polypeptide or fragment/variant thereof via a linker, e.g., a peptide linker or a non-peptide linker. In some embodiments, the spike protein or fragment/variant thereof comprises the RBD. The RBD may include the amino acids 316-542 of the spike protein.

In some embodiments, the fusion polypeptide comprises an amino acid sequence of SEQ ID NO: 5 or an amino acid sequence having at least 75% (e.g., 75%, 80%, 85%, 90%, 95%, 96%, 97%, 98%, 99%) sequence identity to SEQ ID NO: 5.

In some embodiments, a combination of two or more SARS-CoV-2 antigens may be used to detect anti-SARS-CoV-2 antibodies. For example, the combination of SARS-CoV-2 antigens may include a fusion polypeptide (e.g., gp70-RBD) and the S2 subunit. The two or more SARS-CoV-2 antigens may be immobilized on the same solid phase substrate or on separate solid phase substrates. In some embodiments, the SARS-CoV-2 antigen is immobilized on a solid phase substrate either directly or through binding to an immobilized (capture) antibody. For example, in the context of ELISA, the fusion polypeptide, e.g., gp70-RBD, and the S2 subunit may be coated on the same well or on different wells, e.g., directly or through binding to an immobilized (capture) antibody.

In some embodiments, the SARS-CoV-2 antigen may include one or modifications to further improve the correlation between antibody binding specificity and functional activities. For example, such modifications may include modifying RBD antigens in which non-functional targets are mutated or blocked by insertion of glycosylation sites, while retaining the major sites that are targeted by neutralizing antibodies. In some embodiments, such modifications (e.g., in RBD antigens) can be tailored for targeting particular SARS-CoV-2 variants, thereby providing additional information regarding serum specificity for the different variants. In some embodiments, the modifications may include converting the three highlighted Asn residues (e.g., N360, N394, and N388) of RBD to N-linked glycosylation sites by modifying the N+2 residue to Ser or Thr.

As used herein, the term “variant” refers to a first molecule that is related to a second molecule (also termed a “parent” molecule). The variant molecule can be derived from, isolated from, based on or homologous to the parent molecule. For example, the mutant forms of a spike protein are variants of the spike protein. The term variant can be used to describe either polynucleotides or polypeptides.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

In some embodiments, a variant of a SARS-CoV-2 antigen (e.g., spike protein or fragment/variant thereof) antigen may include one or more conservative modifications. The spike protein variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine). The Cas protein with one or more conservative modifications may retain the desired functional properties, which can be tested using the functional assays known in the art.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

In some embodiments, a variant of a SARS-CoV-2 antigen (e.g., spike protein or fragment/variant thereof) can be conjugated or linked to a detectable tag or a detectable marker (e.g., a radionuclide, a fluorescent dye, or an MRI-detectable label). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9.

In some embodiments, the SARS-CoV-2 antigen comprises a detection agent. In some embodiments, the detection agent comprises a biotin moiety. In some embodiments, the SARS-CoV-2 antigen is biotinylated. The term “biotin moiety” refers to an affinity agent that includes biotin or a biotin analog such as desthiobiotin, oxybiotin, 2′-iminobiotin, diaminobiotin, biotin sulfoxide, biocytin, etc. Biotin moieties bind to streptavidin with an affinity of at least 10-8M. A biotin affinity agent may also include a linker, e.g., -LC-biotin, -LC-LC-Biotin, -SLC-Biotin or -PEGn-Biotin where n is 3-12.

The term “streptavidin” refers to both streptavidin and avidin, as well as any variants thereof that bind to biotin with high affinity.

In some embodiments, the detectable tag can be conjugated or linked to the N- and/or C-terminus of a variant of a SARS-CoV-2 antigen (e.g., spike protein or fragment/variant thereof). The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction, etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

In some embodiments, the linker can be a peptide linker and a non-peptide linker. Examples of peptide linkers that can be used in the fusion protein of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS or SGn, where n is an integer from 1 to 10, e.g., 1 2, 3, 4, 5, 6, 7, 8, 9 or 10. In one embodiment, the linker is or comprises a monomer or multimer of repeat of G4S (GGGGS; SEQ ID NO: 11), e.g., (GGGGS)n.

As used herein, the term “non-peptide linker” refers to a biocompatible polymer composed of two or more repeating units linked to each other, in which the repeating units are linked to each other by any non-peptide covalent bond. This non-peptidyl linker may have two ends or three ends. Examples of the non-peptidyl linker may include, without limitation, polyethylene glycol, polypropylene glycol, a copolymer of ethylene glycol with propylene glycol, polyoxyethylated polyol, polyvinyl alcohol, polysaccharide, dextran, polyvinyl ethyl ether, biodegradable polymers such as polylactic acid (PLA) and polylactic-glycolic acid (PLGA), lipid polymers, chitins, hyaluronic acid, and combinations thereof.

In some embodiments, a SARS-CoV-2 antigen can be fused to a fusion partner (e.g., gp-70) through crosslinking with a crosslinking agent, e.g., crosslinker. Crosslinkers are reagents having reactive ends to specific functional groups (e.g., primary amines or sulfhydryls) on proteins or other molecules. Crosslinkers are capable of joining two or more molecules by a covalent bond. Crosslinkers include but are not limited to amine-to-amine crosslinkers (e.g., disuccinimidyl suberate (DSS)), amine-to-sulfhydryl crosslinkers (e.g., N-γ-maleimidobutyryl-oxysuccinimide ester (GMBS)), carboxyl-to-amine crosslinkers (e.g., dicyclo-hexylcarbodiimide (DCC)), sulfhydryl-to-carbohydrate crosslinkers (e.g., N-β-maleimidopropionic acid hydrazide (BMPH)), sulfhydryl-to-sulfhydryl crosslinkers (e.g., 1,4-bismaleimidobutane (BMB)), photoreactive crosslinkers (e.g., N-5-azido-2-nitrobenzoyloxysuccinimide (ANB-NOS)), chemoselective ligation crosslinkers (e.g., NHS-PEG4-Azide).

b. Samples

Samples that can be used in a method according to the present disclosure include any tissue or fluid sample obtainable from a patient, which contains detectable quantities of anti-SARS-CoV-2 antibodies or antigen-binding proteins, under normal or pathological conditions. Generally, levels of anti-SARS-CoV-2 antibodies or antigen-binding proteins in a particular sample obtained from a healthy patient (e.g., a patient not afflicted with a disease associated with SARS-COV-2) will be measured to initially establish a baseline, or standard, level of anti-SARS-CoV-2 antibodies or antigen-binding proteins. This baseline level of anti-SARS-CoV-2 antibodies or antigen-binding proteins can then be compared against the levels of anti-SARS-CoV-2 antibodies or antigen-binding proteins measured in samples obtained from individuals suspected of having a SARS-COV-2-associated condition or symptoms associated with such condition. In some embodiments, the sample comprises a saliva, blood, serum, plasma, cerebrospinal fluid (CSF), peritoneal fluid, or cord blood sample.

As used herein, the term “subject,” “patient,” or “individual” refers to an animal, preferably a mammal, more preferably a human, in need of amelioration, prevention and/or treatment of a disease or disorder such as viral infection. The term includes human subjects who have or are at risk of having SARS-COV-2 infection.

In some embodiments, samples may be obtained from a subject who is asymptomatic. In some embodiments, the subject either has an active infection or has been exposed to the SARS-CoV-2. In some embodiments, the subject has been treated with an anti-inflammatory agent or an antiviral agent or therapy.

As used herein, the term “antiviral agent” or “antiviral “drug” refers to any anti-infective drug or therapy used to treat, prevent, or ameliorate a viral infection in a subject. The term “antiviral drug” includes, but is not limited to ribavirin, oseltamivir, zanamivir, interferon-alpha2b, analgesics, and corticosteroids. In the context of the present disclosure, the viral infections include infection caused by human coronaviruses, including but not limited to, MERS-CoV, HCoV_229E, HCoV_NL63, HCoV-OC43, HCoV_HKU1, SARS-CoV, and SARS-CoV-2.

In some embodiments, the antiviral agent may include: a nucleoside analog, a peptoid, an oligopeptide, a polypeptide, a protease inhibitor, a 3C-like protease inhibitor, an anti-IL6 inhibitor, a papain-like protease inhibitor, or an inhibitor of an RNA dependent RNA polymerase. In some embodiments, the antiviral agent may include acyclovir, gancyclovir, vidarabine, foscarnet, cidofovir, amantadine, ribavirin, trifluorothymidine, zidovudine, didanosine, zalcitabine, tocilizumab, an interferon, or a combination thereof.

In some embodiments, the antiviral agent or therapy comprises a convalescent plasma therapy. Convalescent plasma administration has increasingly become an emergency therapeutic approach during the current COVID-19 pandemic. Due to the present situation, screening of donor plasma must be speedy. On the donor side, the disclosed methods can be used to characterize antibody profiles (e.g., targets, isotypes, functions such as neutralizing or enhancing activity). On the recipient side, the disclosed methods can also be used to determine the effects of convalescent plasma therapy (e.g., anti-SARS-CoV-2 antibody titers).

c. Assays

As understood by a person having ordinary skill in the art, any suitable assay configurations can be used to implement the disclosed methods for detecting an antibody or antigen-binding protein. In some embodiments, the method can be carried out on an apparatus including a conventional lateral flow test strip, e.g., an immunoassay test strip, as an assay medium. In some embodiments, the method can be carried out on a multi-well plate (e.g., a 3-, 6-, 8-, 24-, 96-, 384- or 1536-well plate), and the wells of the plate can further comprise a plurality of distinct assay domains to accommodate measurements of multiple samples from a single subject or from different subjects.

SARS-CoV-2 antigens or fragments/variants thereof may be incorporated into a competitive binding assay or an immunoassay (e.g., ELISA). The SARS-CoV-2 antigens or fragments/variants thereof may be immobilized, either directly or through binding to an immobilized (capture) antibody, onto a variety of supports, such as magnetic or chromatographic matrix particles, the surface of an assay plate (such as microtiter wells), pieces of a solid substrate material, a capture antibody, and the like. An assay plate or strip can be prepared by coating SARS-CoV-2 antigens or fragments/variants thereof in an array on a solid support or by coating with a capture antibody. In one example, the strip can be dipped into the test biological sample and then processed quickly through washes and detection steps to generate a measurable signal, such as a colored spot.

Any solid support known in the art can be used, including but not limited to, solid supports made out of polymeric materials in the forms of wells, tubes or beads. The SARS-CoV-2 antigens or fragments/variants thereof can be bound to the solid support by adsorption, by covalent bonding using a chemical coupling agent, by binding to a capture antibody, or by other means known in the art, provided that such binding does not interfere with the binding ability of the capture proteins. Moreover, if necessary, the solid support can be derivatized to allow reactivity with various functional groups on the proteins. Such derivatization requires the use of certain coupling agents such as, but not limited to, maleic anhydride, N-hydroxysuccinimide, and 1-ethyl-3-(3-dimethylaminopropyl) carbodiimide. In some embodiments, the solid phase substrate may include microparticles, microbeads, magnetic beads, membrane, and an affinity purification column.

In some embodiments, the method is implemented as a competitive binding assay or an immunoassay, e.g., ELISA. In some embodiments, the method may include detecting fluorescence or chemiluminescence. In some embodiments, the competitive binding assay may include detecting the binding of the antibody to the SARS-CoV-2 antigen in the presence of an ACE2 polypeptide or fragment/variant thereof. The ACE2 polypeptide or fragment/variant thereof is capable of binding to the SARS-CoV-2 antigen.

In some embodiments, the step of detecting comprises detecting the antibody or antigen-binding protein bound to the SARS-CoV-2 antigen or the solid phase substrate using a second SARS-CoV-2 antigen that interacts with the antibody or antigen-binding protein. In some embodiments, the second SARS-CoV-2 antigen comprises a detection agent. In some embodiments, the detection agent comprises a biotin moiety. In some embodiments, the second SARS-CoV-2 antigen is biotinylated.

In some embodiments, the method may further include contacting one or more secondary antibodies with the sample. The secondary antibodies are capable of binding anti-SARS-CoV-2 in the sample. For example, a combination of two or more (e.g., 2, 3, 4, 5, 6) secondary antibodies may be used. In some embodiments, a combination of three secondary antibodies is used, with the first secondary antibody reactive against the heavy chain of human IgA, the second secondary antibody reactive against the heavy chain of human IgG, and the third secondary antibody reactive against the heavy chain of human IgG. Some embodiments may also include antibodies reactive with kappa and/or lambda light chains, which are complexed to all forms of heavy chains.

In some embodiments, each of the one or more secondary antibodies (for binding to, e.g., IgG, IgM, IgA heavy chain) may be conjugated to a label, such as a fluorescent label, a chemiluminescent label, a radiolabel, and an enzyme.

The detectable label or reporter molecule can be a radioisotope, such as ³H, ¹³C, ³⁵P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure SARS-COV-2 in a sample include ELISA, radioimmunoassay (RIA), and fluorescence-activated cell sorting (FACS).

### B. SARS-COV-2 Polypeptides and Polynucleotides

This disclosure also provides a polypeptide comprising a spike polypeptide of a SARS-CoV-2 fused to a gp70n polypeptide or fragment/variant thereof. The gp70n fragment used for this purpose is the N-terminal domain of the Murine Leukemia Virus (MuLV) Surface (SU) protein. This corresponds to the receptor-binding region of gp70, the murine leukemia virus surface protein, and is a self-contained structure that folds well, and allows the independent folding of other viral glycoprotein fragments that are fused to the C-terminus of this protein. The spike polypeptide may include the full-length spike glycoprotein or fragments/variants thereof. Similarly, the gp70n polypeptide may include the full-length N-terminal region of the gp70 glycoprotein or fragments/variants thereof.

The spike polypeptide may be fused to the N- or C-terminus of the gp70n polypeptide directly or via a linker (e.g., peptide linker or non-peptide linker). In some embodiments, the polypeptide comprises an amino acid sequence of SEQ ID NO: 5 or an amino acid sequence having at least 75% (e.g., 75%, 80%, 85%, 90%, 95%, 96%, 97%, 98%, 99%) sequence identity to SEQ ID NO: 5.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length. Preferably the peptide fragment can elicit an immune response when used to inoculate an animal. A peptide fragment may be used to elicit an immune response by inoculating an animal with a peptide fragment in combination with an adjuvant, a peptide fragment that is coupled to an adjuvant, or a peptide fragment that is coupled to arsanilic acid, sulfanilic acid, an acetyl group, or a picryl group. A peptide fragment can include a non-amide bond and can be a peptidomimetic.

Also provided in this disclosure are (a) a polynucleotide comprising a polynucleotide sequence that encodes the polypeptide described above; (b) a vector comprising the polynucleotide as described; (c) A host cell comprising the vector as described; (d) a mammalian cell line expressing the polypeptide as described.

In some embodiments, the polynucleotide may include the polynucleotide sequence of SEQ ID NOs: 7-8 or a polynucleotide sequence having at least 75% (e.g., 75%, 80%, 85%, 90%, 95%, 96%, 97%, 98%, 99%) sequence identity to the polynucleotide sequence of SEQ ID NOs: 7-8.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

In some embodiments, the disclosed polypeptide can be encoded by a codon-optimized sequence. For example, the nucleotide sequence encoding the polypeptide may be codon-optimized for expression in a eukaryote or eukaryotic cell. In some embodiments, the codon-optimized polypeptide is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.). In some embodiments, one or more codons (e.g., 1, 2, 3, 4, 5, 10, 15, 20, 25, 50, or more, or all codons) in a sequence encoding the polypeptide corresponds to the most frequently used codon for a particular amino acid. As to codon usage in yeast, reference is made to the online Yeast Genome database available at http://www.yeastgenome.org/community/codonusage.shtml, or Codon selection in yeast, Bennetzen and Hall, J Biol Chem. 1982 Mar. 25; 257(6): 3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al., Nucleic Acids Res. 1989 Jan. 25; 17 (2): 477-98; or Selection on the codon bias of chloroplast and cyanelle genes in different plant and algal lineages, Morton B R, J Mol Evol. 1998 April; 46(4): 449-59.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present disclosure comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector comprises an expression cassette that comprises polynucleotide sequences that encode mutant polypeptides or immunoconjugates or fragments thereof.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### C. Antibodies Against SARS-COV-2 Antigens

This disclosure further provides an isolated antibody or antigen-binding fragment thereof that specifically binds the polypeptide described above. In some embodiments, the isolated antibody or antigen-binding fragment thereof may be isolated from the sample of a subject using a SARS-CoV-2 antigen and fragments/variants thereof as a capture protein.

Accordingly, this disclosure further provides a method of isolating anti-SARS-CoV-2 antibodies comprising: (i) contacting a serum sample from a subject infected with or vaccinated against a SARS-CoV-2 with a SARS-CoV-2 antigen that is immobilized on a surface of a solid phase substrate; (ii) allowing SARS-CoV-2 antibodies in the sample to bind to the SARS-CoV-2 antigen; (iii) optionally washing the solid phase substrate; (iv) releasing bound SARS-CoV-2 antibodies from the solid phase substrate; and (v) collecting the SARS-CoV-2 antibodies released from the solid phase substrate. In some embodiments, the above methods may further include identifying and characterizing the antibodies after the antibodies are isolated.

In some embodiments, the isolated antibody or antigen-binding fragment thereof can be obtained from a subject that has been administered the polypeptide, the polynucleotide or the immunogenic composition, as described above. The antibody is specifically reactive with, e.g., SARS-CoV-2 polypeptides, which have been produced from an immune response elicited by the administration, to a subject, of polynucleotides and/or polypeptides of the present disclosure. Anti-protein/anti-peptide antisera or monoclonal antibodies can be made by standard protocols (See, for example, Antibodies: A Laboratory. Manual ed. by Harlow and Lane (Cold Spring Harbor Press: 1988)). A subject such as a mouse, a hamster, a rabbit, a horse, a human, or non-human primate can be immunized with an immunogenic form of a SARS-CoV-2 polypeptide or polynucleotide, of the present disclosure, encoding an immunogenic form of a SARS-CoV-2 polypeptide. Techniques for conferring immunogenicity on a protein or peptide include conjugation to carriers or other techniques well known in the art. An immunogenic portion of the SARS-CoV-2 polypeptide can be administered in the presence of adjuvant and as part of the compositions described herein. The progress of immunization can be monitored by detecting antibody titers in plasma or serum. Standard ELISA or other immunoassays can be used with the immunogen as antigen to assess the levels of antibodies.

The antibodies may be immunospecific for antigenic determinants of the SARS-CoV-2 polypeptides of the present disclosure, e.g., antigenic determinants of a polypeptide of the present disclosure or a closely related human or non-human mammalian homolog (e.g., 90% homologous and at least about 95% homologous). In some embodiments, the SARS-CoV-2 antibodies do not substantially cross react (i.e., react specifically) with a protein which is, for example, less than 80% percent homologous to a sequence as disclosed. By “not substantially cross react,” it meant that the antibody has a binding affinity for a non-homologous protein which is less than 10 percent, less than 5 percent, or less than 1 percent, of the binding affinity for a protein as disclosed. In an alternative embodiment, there is no cross-reactivity between viral and mammalian antigens.

Any subject can serve as a host for antibody production. Preferred hosts include, but are not limited to, human, non-human primate, mouse, rabbit, horse, goat, donkey, cow, sheep, chickens, cat, dog. Alternatively, antibodies can be produced by cultivation ex vivo of lymphocytes from primed donors stimulated with CD40 resulting in expansion of human B cells (Banchereau et al., Science 251:70 (1991); Zhani et al., J. Immunol. 144:2955-2960, (1990); Tohma et al., J. Immunol. 146:2544-2552 (1991)). Furthermore, an extra in vitro booster step can be used to obtain a higher yield of antibodies prior to immortalization of the cells. See Chaudhuri et al., Cancer Supplement. 73:1098-1104 (1994); Steenbakkers et al. Hum. Antibod. Hybridomas 4:166-173 (1993); Fenano et al., Hum. Antibod. Hybridomas 4:80-85 (1993); Kwekkeboom et al., Immunol. Methods 160:117-127 (1993), which are herein incorporated by reference.

An alternative to human primed donors is to “recreate” or mimic splenic conditions in an immunocompromised animal host, such as the “Severe Combined Immune Deficient” (SCID) mouse. Human lymphocytes are readily adopted by the SCED mouse (hu-SCLD) and produce high levels of immunoglobulins. See Mosier et al., Nature 335:256 (1988); McCune et al., Science 241:1632-1639 (1988). Moreover, if the donor used for reconstitution has been exposed to a particular antigen, a strong secondary response to the same antigen can be elicited in such mice. See Duchosal et al. Nature 355:258-262 (1992).

The term “antibody” as used herein is intended to include fragments thereof that are also specifically reactive with SARS-CoV-2 polypeptides. Antibodies can be fragmented using conventional techniques and the fragments screened for utility in the same manner as described above for whole antibodies. For example, F(ab′)₂ fragments can be generated by treating antibodies with pepsin. The resulting F(ab′)₂ fragment can be treated to reduce disulfide bridges to produce Fab′ fragments. The antibody may be further intended to include bispecific and chimeric molecules having an anti-SARS-CoV-2 portion.

In some embodiments, the antibody is a monoclonal antibody, a polyclonal antibody, a single-chain antibody, an antigen-binding antibody fragment, or a humanized antibody. Both monoclonal and polyclonal antibodies (Ab) directed against SARS-CoV-2 polypeptides or SARS-CoV-2 polypeptide variants, and antibody fragments such as Fab′ and F(ab′), can be used to block the action of SARS-CoV-2 polypeptides and allow the study of the role of a particular SARS-CoV-2 polypeptide as disclosed in the infectious life cycle of the virus and in pathogenesis.

Moreover, the antibodies possess utility as immunoprobes for diagnosis of SARS-CoV-2 infection. This generally comprises taking a sample, e.g., respiratory fluid, of a person suspected of having SARS-CoV-2 infection and incubating the sample with the subject human monoclonal antibodies to detect the presence of SARS-CoV-2 infected cells. This involves directly or indirectly labeling the subject human antibodies with a reporter molecule, which provides for detection of human monoclonal antibody SARS-CoV-2 immune complexes. Examples of known labels include by way of non-limiting example enzymes, e.g., β-lactamase, luciferase, and radio labels. Methods for effecting immunodetection of antigens using monoclonal antibodies are well known in the art.

### D. Immunogenic Compositions

This disclosure further provides an immunogenic composition comprising the polypeptide or the polynucleotide, as described above, and optionally a carrier (e.g., adjuvant). Also provided is a pharmaceutical composition comprising an effective amount of the polypeptide or the polynucleotide described above and optionally a carrier or excipient. In some embodiments, the effective amount of the polypeptide/polynucleotide is effective for inhibition of SARS-CoV-2 fusion with, or entry into, mammalian cells or for treatment of SARS-CoV-2 infection.

The present disclosure further provides a method for generating, enhancing, or modulating a protective and/or therapeutic immune response to SARS-CoV-2 in a subject. The method includes administering to a subject in need of therapeutic and/or preventative immunity one or more of the compositions described herein. In this method, the composition includes an isolated polynucleotide encoding a SARS-CoV-2 polypeptide or a fragment, variant, or derivative thereof and/or an isolated SARS-CoV-2 polypeptide or a fragment, variant, or derivative thereof, for example, a recombinant protein, a purified subunit, or viral vector expressing the protein, as described above. Upon administration of the composition, the SARS-CoV-2 polypeptide or a fragment, variant, or derivative thereof is expressed in the subject in a therapeutically or prophylactically effective amount.

In some embodiments, the polynucleotide or polypeptide compositions of the present disclosure may be administered to a subject where the subject is used as an in vivo model to observe the effects of individual or multiple SARS-CoV-2 polypeptides in vivo. This approach would not only eliminate the species-specific barrier to studying SARS-CoV-2, but would allow for the study of the immunopathology of SARS-CoV-2 polypeptides as well as SARS-CoV-2 polypeptide specific effects without using infectious SARS-CoV-2 virus. An in vivo vertebrate model of SARS-CoV-2 infection would be useful, for example, in developing treatments for one or more aspects of SARS-CoV-2 infection by mimicking those aspects of infection without the potential hazards associated with handling the infectious virus

One or more compositions of the present disclosure are utilized in a “prime boost” regimen. An example of a “prime boost” regimen may be found in Yang, Z. et al., J. Virol. 77:799-803 (2002). In these embodiments, one or more polynucleotide immunogenic compositions (e.g., vaccine compositions) of the present disclosure are delivered to a subject, thereby priming the immune response of the subject to SARS-CoV-2, and then a second immunogenic composition is utilized as a boost vaccination. One or more immunogenic compositions of the present disclosure are used to prime immunity, and then a second immunogenic composition, e.g., a recombinant viral vaccine or vaccines, a different polynucleotide vaccine, or one or more purified subunit isolated SARS-CoV-2 polypeptides or fragments, variants or derivatives thereof is used to boost the anti-SARS-CoV-2 immune response.

In one embodiment, a priming composition and a boosting composition are delivered to a subject in separate doses and vaccinations. For example, a single composition may comprise one or more polynucleotides encoding SARS-CoV-2 protein(s), fragment(s), variant(s), or derivative(s) thereof and/or one or more isolated SARS-CoV-2 polypeptide(s) or fragment(s), variant(s), or derivative(s) thereof as the priming component. The polynucleotides encoding the SARS-CoV-2 polypeptides fragments, variants, or derivatives thereof may be contained in a single plasmid or viral vector or in multiple plasmids or viral vectors. At least one polynucleotide encoding a SARS-CoV-2 protein and/or one or more SARS-CoV-2 isolated polypeptide can serve as the boosting component. In this embodiment, the compositions of the priming component and the compositions of the boosting component may be contained in separate vials. In one example, the boosting component is administered approximately 1 to 6 months after administration of the priming component.

In one embodiment, a priming composition and a boosting composition are combined in a single composition or single formulation. For example, a single composition may comprise an isolated SARS-CoV-2 polypeptide or a fragment, variant, or derivative thereof as the priming component and a polynucleotide encoding a SARS-CoV-2 protein as the boosting component. In this embodiment, the compositions may be contained in a single vial where the priming component and boosting component are mixed together. In general, because the peak levels of expression of protein from the polynucleotide do not occur until later (e.g., 7-10 days) after administration, the polynucleotide component may provide a boost to the isolated protein component. Compositions comprising both a priming component and a boosting component are referred to herein as “combinatorial vaccine compositions” or “single formulation heterologous prime-boost vaccine compositions.” In addition, the priming composition may be administered before the boosting composition, or even after the boosting composition, if the boosting composition is expected to take longer to act.

In another embodiment, the priming composition may be administered simultaneously with the boosting composition, but in separate formulations where the priming component and the boosting component are separated. In certain embodiments, one or more compositions of the present disclosure are delivered to a subject (e.g., vertebrate) by methods described herein, thereby achieving an effective therapeutic and/or an effective preventative immune response. More specifically, the compositions may be administered to any tissue of a vertebrate, including, but not limited to, muscle, skin, brain tissue, lung tissue, liver tissue, spleen tissue, bone marrow tissue, thymus tissue, heart tissue, e.g., myocardium, endocardium, and pericardium, lymph tissue, blood tissue, bone tissue, pancreas tissue, kidney tissue, gall bladder tissue, stomach tissue, intestinal tissue, testicular tissue, ovarian tissue, uterine tissue, vaginal tissue, rectal tissue, nervous system tissue, eye tissue, glandular tissue, tongue tissue, and connective tissue, e.g., cartilage.

Compositions of the present disclosure may include various adjuvants, salts, excipients, delivery vehicles and/or auxiliary agents as are disclosed, e.g., in U.S. Patent Application Publication 2002/0019358, published Feb. 14, 2002, which is incorporated herein by reference in its entirety.

In some embodiments, the adjuvant may include aluminum hydroxide, lipid A, killed bacteria, polysaccharide, mineral oil, Freund's incomplete adjuvant, Freund's complete adjuvant, aluminum phosphate, iron, zinc, a calcium salt, acylated tyrosine, an acylated sugar, a cationically derivatized polysaccharide, an anionically derivatized polysaccharide, a polyphosphazene, a biodegradable microsphere, a monophosphoryl lipid A, and quil A.

The ability of an adjuvant to increase the immune response to an antigen is typically manifested by a significant increase in immune-mediated protection. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or the response into a primarily cellular, or T cell response. In certain embodiments, the compositions may be administered in the absence of one or more adjuvants, transfection facilitating materials or auxiliary agents.

Compositions of the present disclosure can be formulated according to known methods. Suitable preparation methods are described, for example, in Remington's Pharmaceutical Sciences, 16th Edition, A. Osol, ed., Mack Publishing Co., Easton, PA (1980), and Remington's Pharmaceutical Sciences, 19th Edition, A. R. Gennaro, ed., Mack Publishing Co., Easton, PA (1995), both of which are incorporated herein by reference in their entireties. Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives.

### E. Kits

In another aspect, this disclosure also provides a kit comprising a first detection reagent comprising the polypeptide as described above. In some embodiments, the polypeptide comprises a spike polypeptide fused to a gp70 polypeptide. In some embodiments, the polypeptide comprises an amino acid sequence of SEQ ID NO: 5 or an amino acid sequence having at least 75% sequence identity to SEQ ID NO: 5.

In some embodiments, the kit further comprises a second detection reagent comprising the polypeptide described above. In some embodiments, the polypeptide contained in the second detection reagent comprises a detection label. In some embodiments, the detection label comprises a biotin moity. In some embodiments, the polypeptide is biotinylated.

A composition described above can be provided in a kit. In one embodiment, the kit includes (a) a container that contains the composition, and optionally (b) informational material. The informational material can be descriptive, instructional, marketing or other material that relates to the methods described herein and/or the use of the compositions for therapeutic benefit. In an embodiment, the kit includes also includes an additional therapeutic agent (e.g., antiviral agent, anti-inflammatory agent). For example, the kit may include a first container that contains the composition and a second container for the additional therapeutic agent.

The informational material of the kits is not limited in its form. In one embodiment, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

In addition to the composition, the kit can include other ingredients, such as a solvent or buffer, a stabilizer, or a preservative. The composition can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit.

In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition, e.g., a syringe or other suitable delivery device. The device can be provided pre-loaded with one or both of the agents or can be empty, but suitable for loading.

Also provided in this disclosure are kits for detecting anti-SARS-CoV-2 antibodies or antigen-binding proteins in a sample. The kits can be used for risk classification of a subject or group of subjects having or at risk of SARS-CoV-2 infections. Accordingly, a kit of the present disclosure may include one or more reagents for detecting the presence of anti-SARS-CoV-2 antibodies or the presence of the SARS-CoV-2 in a subject. For example, the kit may include one or more SARS-CoV-2 antigens (e.g., RBD, gp70-RBD, S1 subunit, S2 subunit, nucleocapsid protein, fragments/variants thereof or combinations thereof). In another example, the kit may also include the antibody, as described above.

In some embodiments, the kit may also include one or more secondary antibodies (for detecting, e.g., IgA, IgG, IgM heavy chain) capable of binding to anti-SARS-CoV-2 antibodies. The secondary antibodies may be optionally labeled with a detectable label, such as a fluorophore, radioactive moiety, enzyme, biotin/avidin label, chromophore, chemiluminescent label, a tag, or the like. The kit can include reagents for labeling the proteins, antibodies or reagents for detecting the antibodies (e.g., detection antibodies) and/or for labeling analytes or reagents for detecting the antibodies.

The kit can comprise a calibrator or control, e.g., purified and optionally lyophilized, antigens, antibodies or combinations thereof. The kit can include at least one container (e.g., tube, microtiter plates or strips, which can be already coated with a suitable antigen or antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The antibodies, calibrators and/or controls can be provided in separate containers or pre-dispensed into an appropriate assay format, for example, into microtiter plates.

If desired, the kit can further comprise one or more components, alone or in further combination with instructions, for assaying the test sample for another analyte, which can be a biomarker, such as a biomarker of sepsis, as mentioned above.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

If desired, the kit can contain a solid phase support, such as a magnetic particle, bead, test tube, microtiter plate, cuvette, membrane, scaffolding molecule, film, filter paper, a quartz crystal, disc or chip. The kit may also include a detectable label that can be or is conjugated to an antigen or an antibody. The detectable label can, for example, be a direct label, which may be an enzyme, oligonucleotide, nanoparticle chemiluminophore, fluorophore, fluorescence quencher, chemiluminescence quencher, or biotin. Kits may optionally include any additional reagents needed for detecting the label.

Kits can contain instructions for their use. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. The instructions also can include instructions for generating a standard curve or a reference standard for purposes of quantifying a target (e.g., anti-SARS-CoV-2 antibodies).

### F. Methods of Use

In another aspect, this disclosure further provides a method of treating or inhibiting a SARS-CoV-2 infection. The method includes administering to a mammal in need a therapeutically effective amount of the polypeptide, the polynucleotide, the composition or the isolated antibody or antigen-binding fragment thereof, as described above.

This disclosure also provides a method for eliciting a detectable immune response to the spike polypeptide or fragment/variant thereof. Additionally provided is a method of vaccinating a subject. These methods comprise administering to a mammal the polypeptide, the polynucleotide, and/or the composition, as described above.

In some embodiments, the antibody and the polynucleotide or polypeptide of the present disclosure can be administered simultaneously (at the same time) or subsequent to the administration of the isolated antibodies, thereby providing both immediate and long-lasting protection.

The antibody can be used as therapeutic and prophylactic agents to treat or prevent SARS-CoV-2 infection by passive antibody therapy. In general, this comprises administering a therapeutically or prophylactically effective amount of the monoclonal or polyclonal antibodies to a susceptible subject or one exhibiting SARS-CoV-2 infection. An effective dosage amount will range from about 50 to 20,000 μg/kg, and from about 100 to 5,000 μg/kg. However, suitable dosages may vary depending on factors, such as the condition of the treated host, weight, etc. Suitable effective dosages may be determined by those skilled in the art.

The monoclonal or polyclonal antibodies may be administered by any mode of administration suitable for administering antibodies. Typically, the subject antibodies will be administered by injection, e.g., intravenous, intramuscular, or intraperitoneal injection (as described previously), or aerosol. Aerosol administration is preferred if the subjects treated comprise newborn infants. Formulation of a composition comprising the antibody in pharmaceutically acceptable form may be affected by known methods, using known pharmaceutical carriers and excipients. Suitable earners and excipients include by way of non-limiting example buffered saline and bovine serum albumin.

For example, the compositions may be administered to any internal cavity of a subject (e.g., vertebrate), including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, the ocular cavities, the lumen of a duct of a salivary gland or a liver. When the compositions are administered to the lumen of a duct of a salivary gland or liver, the desired polypeptide is expressed in the salivary gland and the liver such that the polypeptide is delivered into the bloodstream of the subject from each of the salivary gland or the liver. Certain modes for administration to secretory organs of a gastrointestinal system using the salivary gland, liver, and pancreas to release a desired polypeptide into the bloodstream are disclosed in U.S. Pat. Nos. 5,837,693 and 6,004,944, both of which are incorporated herein by reference in their entireties.

In some embodiments, the compositions are administered to muscle, either skeletal muscle or cardiac muscle, or to lung tissue. Specific but non-limiting modes for administration to lung tissue are disclosed in Wheeler, C. J., et al., Proc. Natl. Acad. Sci. USA 93:11454-11459 (1996), which is incorporated herein by reference in its entirety.

According to the disclosed methods, a polypeptide, polynucleotide or composition, or an antibody can be administered by intramuscular (i.m.), subcutaneous (s.c), or intrapulmonary routes. Other suitable routes of administration include, but are not limited to intratracheal, transdermal, intraocular, intranasal, inhalation, intracavity, intravenous (i.v.), intraductal (e.g., into the pancreas) and intraparenchymal (i.e., into any tissue) administration. Transdermal delivery includes, but is not limited to intradermal (e.g., into the dermis or epidermis), transdermal (e.g., percutaneous), and transmucosal administration (i.e., into or through skin or mucosal tissue). Intracavity administration includes, but is not limited to administration into oral, vaginal, rectal, nasal, peritoneal, or intestinal cavities as well as, intrathecal (i.e., into spinal canal), intraventricular (i.e., into the brain ventricles or the heart ventricles), inraatrial (i.e., into the heart atrium) and sub arachnoid (i.e., into the sub arachnoid spaces of the brain) administration.

Any mode of administration can be used so long as the mode results in the expression of the desired peptide or protein, in the desired tissue, in an amount sufficient to generate an immune response to SARS-CoV-2 and/or to generate a prophylactically or therapeutically effective immune response to SARS-CoV-2 in a subject in need of such response.

Administration means may include needle injection, catheter infusion, biolistic injectors, particle accelerators (e.g., “gene guns” or pneumatic “needleless” injectors) Med-E-Jet (Vahlsing, H., et al., J. Immunol. Methods 171:11-22 (1994)), Pigjet (Schrijver, R., et al., Vaccine 15:1908-1916 (1997)), Biojecior (Davis, H., et al., Vaccine 12:1503-1509 (1994); Gramzinski, R., et al., Mol. Med. 4:109-118 (1998)), AdvantaJet (Linmayer, I., et al., Diabetes Care 9:294-291 (1986)), Medi-jector (Martins, J., and Roedl, E. J. Occup. Med. 27:821-824 (1979)), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, Y., et al., Life Sciences 65:2193-2203 (1999)) or topical applications during surgery. Certain modes of administration are intramuscular needle-based injection and pulmonary application via catheter infusion. Energy-assisted plasmid delivery (EAPD) methods may also be employed to administer the compositions of the disclosure. One such method involves the application of brief electrical pulses to injected tissues, a procedure commonly known as electroporation. See generally Mir, L. M. et al., Proc. Natl. Acad. Sci USA 96:4262-1 (1999); Hartikka, J. et al., Mol. Ther. 4:401-15 (2001); Mathiesen, I., Gene Ther. 5:508-14 (1999); Rizzuto G. et al., Hum. Gen. Ther. 77:1891-900 (2000). Each of the references cited in this paragraph is incorporated herein by reference in its entirety.

Determining an effective amount of one or more compositions depends upon a number of factors including, for example, the antigen being expressed or administered directly, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian.

### G. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2, and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen (e.g., a spike or S protein of SARS-CoV-2 virus). It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L}, and C_{H}I domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3^{rd} ed. 1993)); (iv) a Fd fragment consisting of the V_{H} and C_{H}I domains; (v) a Fv fragment consisting of the V_{L} and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds to a spike or S protein of SARS-CoV-2 virus is substantially free of antibodies that specifically bind antigens other than the neuraminidase). An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “antigen-binding protein” as used herein means any protein that specifically binds a specified target antigen, such as those described above. The term antigen-binding protein encompasses intact antibodies that comprise at least two full-length heavy chains and two full-length light chains, as well as derivatives, variants, fragments, and mutations thereof. Examples also include antibody mimetics.

As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901 and 6,348,584, the contents of each of which are herein incorporated by reference in their entirety). In some embodiments, antibody mimetics may include those known in the art including, but not limited to, Adnectins, Affibodies, Affilins, Affimers, Affitins, Alphabodies, Anticalins, Aptamers, Armadillo repeat protein, based scaffolds, Atrimers, Avimers, Centyrins, DARPins, Fynomers, Knottins, Kunitz domain peptide, Monobodics, and Nanofitin. In other embodiments, antibody mimetics may include one or more non-peptide regions.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody), and the constant region sequence or Fc is derived from a different source (e.g., a different antibody, such as an IgG, IgA2, IgD, IgE or IgM antibody).

This disclosure encompasses isolated or substantially purified nucleic acids, peptides, polypeptides or proteins. An “isolated” nucleic acid, DNA or RNA molecule or an “isolated” polypeptide is a nucleic acid, DNA molecule, RNA molecule, or polypeptide that exists apart from its native environment and is therefore not a product of nature. An isolated nucleic acid, DNA molecule, RNA molecule or polypeptide may exist in a purified form or may exist in a non-native environment such as, for example, a transgenic host cell. A “purified” nucleic acid molecule, peptide, polypeptide or protein, or a fragment thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein, peptide or polypeptide that is substantially free of cellular material includes preparations of protein, peptide or polypeptide having less than about 30%, 20%, 10%, or 5% (by dry weight) of contaminating protein. When the protein of the present disclosure, or biologically active portion thereof, is recombinantly produced, preferably culture medium represents less than about 30%, 20%, 10%, or 5% (by dry weight) of chemical precursors or non-protein-of-interest chemicals.

The term “recombinant,” as used herein, refers to antibodies or antigen-binding fragments thereof created, expressed, isolated or obtained by technologies or methods known in the art as recombinant DNA technology, which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary, to join two protein-coding regions in the same reading frame.

As used herein, the term “promoter” or “regulatory sequence” refers to a nucleic acid sequence that is required for expression of a gene product operably linked to the promoter/regulatory sequence. In some instances, this sequence may be the core promoter sequence, and in other instances, this sequence may also include an enhancer sequence and other regulatory elements that are required for expression of the gene product. The promoter or regulatory sequence may, for example, be one that expresses the gene product in a tissue-specific manner. An “inducible” promoter is a nucleotide sequence that, when operably linked with a polynucleotide that encodes or specifies a gene product, causes the gene product to be produced in a cell substantially only when an inducer that corresponds to the promoter is present in the cell.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acid substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

The term “specifically binds,” or “binds specifically to,” or the like, refers to an antibody that binds to a single epitope, e.g., under physiologic conditions, but which does not bind to more than one epitope. Accordingly, an antibody that specifically binds to a polypeptide will bind to an epitope that present on the polypeptide, but which is not present on other polypeptides. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to a spike or S protein of the SARS-CoV-2 virus.

Preferably, the antibody binds to a spike or S protein with “high affinity,” namely with a KD of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less, as determined by surface plasmon resonance, e.g., BIACORE. The term “does not substantially bind” to a protein or cells, as used herein, means does not bind or does not bind with a high affinity to the protein or cells, i.e., binds to the protein or cells with a KD of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “Kassoc” or “Ka,” as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a BIACORE system.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In some embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, In some embodiments, may have specific three-dimensional structural characteristics and/or specific charge characteristics. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from a spike or S protein are tested for reactivity with a given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

An “antigen” refers to a molecule containing one or more epitopes (either linear, conformational or both) that will stimulate a host's immune system to make a humoral and/or cellular antigen-specific response. The term is used interchangeably with the term “immunogen.” Normally, an epitope will include between about 3-15, generally about 5-15 amino acids. A B-cell epitope is normally about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as 9, 10, 12 or 15 amino acids. The term “antigen” denotes both subunit antigens (i.e., antigens which are separate and discrete from a whole organism with which the antigen is associated in nature), as well as killed, attenuated or inactivated bacteria, viruses, fungi, parasites or other microbes as well as tumor antigens, including extracellular domains of cell surface receptors and intracellular portions that may contain T-cell epitopes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide that expresses an antigen or antigenic determinant in vivo, such as in gene therapy and DNA immunization applications, is also included in the definition of antigen herein.

The term “epitope mapping” refers to the process of identification of the molecular determinants for antibody-antigen recognition.

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binds to the same epitope” with reference to two or more antibodies means that the antibodies bind to the same, overlapping or encompassing continuous or discontinuous segments of amino acids. Those of skill in the art understand that the phrase “binds to the same epitope” does not necessarily mean that the antibodies bind to or contact exactly the same amino acids. The precise amino acids that the antibodies contact can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

An “immunogenic composition” as used herein refers to a composition that comprises an antigenic molecule where administration of the composition to a subject results in the development in the subject of a humoral and/or a cellular immune response to the antigenic molecule of interest. The immunogenic composition can be introduced directly into a recipient subject, such as by injection, inhalation, oral, intranasal or any other parenteral, mucosal or transdermal (e.g., intra-rectally or intra-vaginally) route of administration.

As used herein, the term “immune response” refers to a biological response within a subject (e.g., vertebrate) against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

The term “detectable label” as used herein refers to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof that is capable of exhibiting fluorescence in the detectable range.

As used herein, the term “disease” is intended to be generally synonymous and is used interchangeably with the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, the term “treating” or “treatment” of any disease or disorder refers, in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically (e.g., stabilization of a discernible symptom), physiologically (e.g., stabilization of a physical parameter), or both. In yet another embodiment, “treating” or “treatment” refers to preventing or delaying the onset or development or progression of the disease or disorder (e.g., SARS-CoV-2 infection).

The terms “prevent,” “preventing,” “prevention,” “prophylactic treatment,” and the like refer to reducing the probability of developing a disorder or condition (e.g., SARS-CoV-2 infection) in a subject who does not have, but is at risk of or susceptible to developing a disorder or condition. The term includes prevention of spread of infection in a subject exposed to the virus or at risk of having SARS-CoV-2 infection.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to body weight. Thus, a dose which is expressed as [g, mg, or other unit]/kg (or g, mg etc.) usually refers to [g, mg, or other unit] “per kg (or g, mg etc.) bodyweight,” even if the term “body weight” is not explicitly mentioned.

As used herein, the term “composition” or “pharmaceutical composition” refers to a mixture of at least one component useful within this disclosure with other components, such as carriers, stabilizers, diluents, dispersing agents, suspending agents, thickening agents, and/or excipients. The pharmaceutical composition facilitates administration of one or more components of this disclosure to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of this disclosure and are physiologically acceptable to the subject. Supplementary active compounds may also be incorporated into the compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

The terms “priming” or “primary” and “boost” or “boosting” as used herein may refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously.

“Sample,” “test sample,” and “patient sample” may be used interchangeably herein. The sample can be a sample of saliva, serum, urine, plasma, amniotic fluid, cerebrospinal fluid, peritoneal fluid, cord blood sample, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., COVID-19), and/or who has been diagnosed with inflammatory disorders. In some embodiments, the subject is a mammal. In some embodiments, the subject is human.

As used herein, a “vertebrate” in need of therapeutic and/or preventative immunity” refers to an individual for whom it is desirable to treat, i.e., to prevent, cure, retard, or reduce the severity of COVID-19 symptoms, and/or result in no worsening of COVID-19 over a specified period of time. Vertebrates to treat and/or vaccinate include humans, apes, monkeys (e.g., owl, squirrel, cebus, rhesus, African green, patas, cynomolgus, and Cercopithecus), orangutans, baboons, gibbons, and chimpanzees, dogs, wolves, cats, lions, and tigers, horses, donkeys, zebras, cows, pigs, sheep, deer, giraffes, bears, rabbits, mice, ferrets, seals, whales, ducks, geese, terns, shearwaters, gulls, turkeys, chickens, quail, pheasants, geese, starlings, and budgerigars.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of this disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither, or both limits are included in the smaller ranges is also encompassed within this disclosure, subject to any specifically excluded limit in the stated range. Where the stated range includes one or both of the limits, ranges excluding either or both of those included limits are also included in this disclosure.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### H. EXAMPLES

### Example 1

This example describes the materials and methods used in the subsequent examples below.

### ELISA Buffers

Coating Buffer: Mix 1.6 g sodium carbonate+3.0 g sodium bicarbonate in 500 ml ddH₂O. pH: 9.6-9.8. Store at 4° C.

Wash Buffer: Add 0.5 ml Tween 20 (SIGMA, Cat #P1379-1L) in 1 liter of 1×PBS (made from 10×PBS diluted in ddH2O) (CORNING, Cat #46-013 CM) (Final Tween 20 concentration: 0.05%). Add 0.02% NaN₃ (FISHER CHEMICAL, Cat #S227I-100). Store at 4° C.

Blocking Buffer: 2% BLOTTO: Mix 10 g non-fat powdered milk (Nestle, Carnation dry non-fat milk)+/−0.25 ml Tween 20 (SIGMA, Cat #P1379-1L) (Final Tween 20 concentration: 0.05%) into 500 ml PBS+0.02% NaN₃. Store at 4° C. for one week.

### Secondary Antibody Cocktail Preparation

A mixture of three secondary antibodies was used. The secondary antibodies were obtained from JACKSON IMMUNORESEARCH LABORATORIES in powder form.
A) Alkaline Phosphatase-Conjugated goat anti-human Fc α (VWR, Cat #109-055-011)
B) Alkaline Phosphatase-Conjugated goat anti-human Fc γ (VWR, Cat #109-055-098)
C) Alkaline Phosphatase-Conjugated goat anti-human Fc μ (VWR, Cat #109-055-129)

Each antibody was resuspended in 0.5 ml ddH₂O. To the solubilized antibody, 0.5 ml of 100% glycerol was added to obtain 1 mg/ml of antibody stock solution in 50% glycerol. Each antibody was stored in aliquots at −80° C. for prolonged storage.

For working antibody cocktail: Equal volume from each antibody was mixed, and small aliquots of antibody cocktail were stored at −20° C. The working stock was diluted to 1:2,000 in a 2% Blotto blocking buffer for ELISA assay.

### Developing Buffer

Mix 48.5 ml diethanolamine (SIGMA, Cat #D8885-1 kg)+24.5 mg MgCl₂·6H2O (SIGMA, Cat #M9272-500G) into 500 ml ddH₂O. Adjust the pH to 9.8 with HCl. Store at 4° C.

### Color Developing Substrate Preparation

Dissolve one tablet of phosphate substrate (SIGMA, Cat #S0942-200TAB) in 5 ml of developing buffer. Prepare a fresh color-developing substrate before each use, precool it for 30 minutes, and protect it from light before adding it to the plates.

### Equipment

A BSL2 plus laboratory equipped with a Hamilton Microlab STARlet liquid handler, a BioTek EL406 combination washer dispenser, and a Synergy Neo2 BioTek microplate reader. Both BioTek instruments are integrated with an automated BioStack™ 4 Microplate Stacker.

### ELISA Protocol

Plate Coating with Antigens

For screening, a mix of two antigens S2 and RBD used in the ELISA plate. Antigens were diluted in coating buffer. 100 ng of each antigen (100 ng recombinant S2 obtained in E. coli from RAY BIOTECH 230-01103-100+100 ng recombinant RBD-gp70 fusion protein described in this disclosure) was used to coat ELISA plates (THERMO FISHER SCIENTIFIC, Cat #439454) in total 50 μl of coating buffer in each well. ELISA plates are covered with a lid and incubated at 4° C. overnight.

Before proceeding with the following steps, all reagents were brought to room temperature.

### Blocking Reaction

Prior to blocking, plates coated overnight with antigen were washed three times with wash buffer using an automated ELISA washer. 100 μl of 2% BLOTTO buffer (+/−0.05% Tween 20) was added to each well, and plates were covered with a lid and incubated at 37° C. for 30 minutes.

### Antigen-Antibody Reaction

After blocking, ELISA plates were washed three times with 100 μl wash buffer using an automated ELISA washer. Heat-inactivated patient serum (patient samples are processed in BSL2+ facility, below) was used at 1:25 dilution made in 2% BLOTTO buffer (5 μl serum in 120 μl). 50 μl of diluted serum was added to each well and tested in duplicate. ELISA plates were covered with a lid and incubated at 37° C. for 60 min.

### Control Sera

Antibody-positive COVID-19 patient serum was used as positive control, and commercial serum (Human serum type AB (male) from SIGMA, Cat #H-4522-20 ml) was used as negative control for plate-to-plate variation. Both positive and negative control sera were diluted in 2% BLOTTO buffer (1:25, 1:50, 1:100), and 50 μl volume of each was added to the wells of the plates. They were tested in duplicate on each plate.

### Other Plate Controls

One control well without primary antibody and one control well without primary antibody and antigen were included in each plate.

### Secondary Antibody Reaction

Before secondary antibody reaction, ELISA plates are washed three times with wash buffer using the automated ELISA washer. 50 μl of diluted secondary antibody cocktail (1:2,000) will be added to each well.

ELISA plates were covered with a lid and incubated at 37° C. for 30 minutes. After incubation, plates were washed three times with wash buffer using the automated washer. Prior to discarding, tubes and tips were treated with concentrated germicidal bleach.

Color Development with Alkaline Phosphatase Substrate

50 μl of color developing substrate was added to each well, and plates were covered with a lid and incubated at 37° C. for 30 minutes. Read plates at OD₄₀₅ using ELISA Plate reader (Synergy H1 Microplate Reader, BIOTEK). After the assay, plates are treated with bleach and discarded as regulated medical waste.

### Handling of Patient Samples in BSL2+ Facility

Patient blood samples were processed in BSL2+ facility designated for COVID-19 work. Blood samples were centrifuged within the BSL2+ facility using sealed centrifuge buckets. Buckets were opened inside the biosafety cabinets. Serum was collected, distributed in aliquots, and stored at −80° C. Working aliquots were heat-inactivated at 56° or 60° C. for 60 minutes (Amanat, et al., medRxiv 2020.03.17.20037713), prior to dilution with 2% BLOTTO buffer. Heat inactivation was performed in a 96-well PCR machine (Eppendorf Realplex) using the 0.2 ml PCR tubes (APPLIED BIOSYSTEMS, Cat #4316567), which were sealed with an optical cap (APPLIED BIOSYSTEMS, Cat #4323032). Before discarding, tubes and tips were treated with concentrated germicidal bleach. Researchers in the BSL2+ facility followed BSL3 working practices and used double gloves, mask, apron, sleeves, and face shield.

TABLE 2
Example ELISA plate scheme

| | DUPLICATE | | DUPLICATE | | DUPLICATE | | DUPLICATE | | DUPLICATE | | | |
|---|---|---|---|---|---|---|---|---|---|---|---|---|
| | SAMPLES | | SAMPLES | | SAMPLES | | SAMPLES | | SAMPLES | | CONTROLS | |
| | 1 | 2 | 3 | 4 | 5 | 6 | 7 | 8 | 9 | 10 | 11 | 12 |
| A | 1 | 1 | 9 | 9 | 17 | 17 | 25 | 25 | 33 | 33 | 25 | 25 |
| | | | | | | | | | | | POS | POS |
| B | 2 | 2 | 10 | 10 | 18 | 18 | 26 | 26 | 34 | 34 | 50 | 50 |
| | | | | | | | | | | | POS | POS |
| C | 3 | 3 | 11 | 11 | 19 | 19 | 27 | 27 | 35 | 35 | 100 | 100 |
| | | | | | | | | | | | POS | POS |
| E | 4 | 4 | 12 | 12 | 20 | 20 | 28 | 28 | 36 | 36 | 25 | 25 |
| | | | | | | | | | | | NEG | NEG |
| E | 5 | 5 | 13 | 13 | 21 | 21 | 29 | 29 | 37 | 37 | 50 | 50 |
| | | | | | | | | | | | NEG | NEG |
| F | 6 | 6 | 14 | 14 | 22 | 22 | 30 | 30 | 38 | 38 | 100 | 100 |
| | | | | | | | | | | | NEG | NEG |
| G | 7 | 7 | 15 | 15 | 23 | 23 | 31 | 31 | 39 | 39 | NO | NO |
| | | | | | | | | | | | PRIM | PRIM |
| H | 8 | 8 | 16 | 16 | 24 | 24 | 32 | 32 | 40 | 40 | NO | NO |
| | | | | | | | | | | | ANTIG | ANTIG |

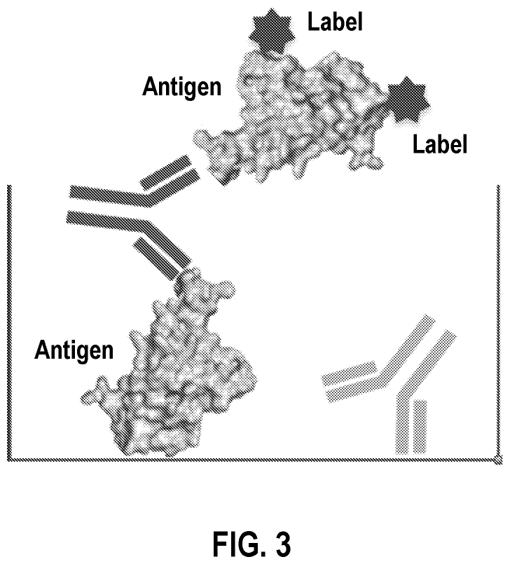

An example of the ELISA plate scheme is provided in FIGS. 3A and 3B. An alternative to the above ELISA plate scheme is to leave the external rows (A and H) and columns (1 and 12) empty to reduce the risk of errors generated from uneven temperature. The reduced number of useable wells can be overcome by full automation and large scale ELISA assays.

### Example 2

### Advantages of the Gp70-RBD Antigen Over Standard Forms in Seroprevalence Assays

The serological assay uses an ELISA based method to identify whether any person or population has been exposed to SARS-CoV-2 by looking at their immune response. The presence or absence of antibodies against the specific viral antigens (e.g., S2 subunit or fragment thereof, S1 subunit or fragment thereof, Receptor Binding Domain (RBD) in S1 subunit or fragment thereof, or secondary antigens such as nucleocapsid (NC) or fragment thereof) were used as an indicator. Patient samples, such as patient's saliva/blood/serum/plasma, were used for the analysis. The presence of antibodies against SARS-CoV-2 antigens in the saliva/blood/serum/plasma of an individual will indicate that either individual has an active infection or had been exposed to SARS-CoV-2 in the past. The high-throughput nature of this assay is used to study the extent of past/present infection in large populations.

One important aspect of the disclosure is the novel viral antigen to be used for detecting the presence of antibodies or antigen-binding proteins that bind specifically to one or more SARS-CoV-2 antigens. The novel viral antigen includes a unique fusion protein system comprising a SARS-CoV-2 antigen (e.g., RBD) fused to a fragment of a foreign viral glycoprotein, such as gp70. The fusion protein system may also facilitate improved protein expression and folding as well as glycosylation. The presence of the gp70 carrier domain serves as a large protein tag that facilitates the purification of the antigen and the capture and presentation of the antigen on the surface of the ELISA plate. In one example, the fusion protein system also includes an affinity tag to facilitate protein purification and a specific epitope recognized by a rat monoclonal antibody that is used for additional characterizations.

To examine whether the use of the gp70-fusion form of RBD antigen is advantageous over standard forms, gp70-RBD (RBD fused to the C-terminus of gp70) was compared to a standard RBD-BEI antigen (or RBD-His6). The purified RBD-His6 antigen was expressed from a vector obtained from the NIH under BEI Repository, catalog #NR-52306 (https://www.niaid.nih.gov/research/bei-resources-repository). This vector was provided to BEI by Dr. F. Krammer at Mt. Sinai, and the antigen produced from this vector is currently being used in the FDA-EAU approved assay.

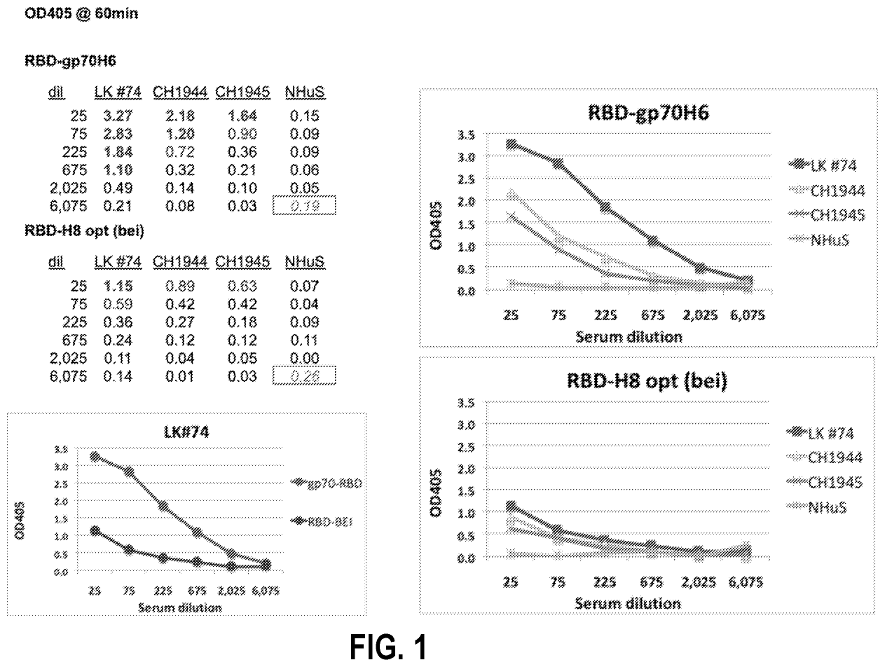

The sensitivity of the two RBD forms as antigens was tested for the detection and titration of antibodies in SARS-CoV-2 convalescent patient sera. The sera used in this assay included one high titer serum (LK #74) and two low titer sera (CH-1944 and CH-1945) along with one normal human serum control (FIG. 1). The results showed a clear advantage of the gp70-RBD antigen over the RBD-BEI antigen for all three positive sera. For the high-titer serum, the signal obtained for the gp70-RBD antigen at the 1:675 dilution was equivalent to that at the 1:25 dilution for the RBD-His6 antigen (1.15)—an apparent 27-fold increase of sensitivity for the gp70-RBD antigen.

### Example 3

In addition to its use as a marker for SARS-CoV-2 infection in serodetection assays, the RBD antigens (e.g., gp70-RBD antigen) are also useful as markers, for example, in a competition assay, to detect the ability of antibodies in patient sera to block the interaction between an RBD antigen and soluble ACE2, the soluble form of the SARS-CoV-2 primary cell-surface receptor. Binding with the ACE2 receptor is known to mediate viral entry into the target cells and is believed to be a required step in infection. Inhibition of the interaction between ACE2 and the virus is thought to be the main mechanism of protective immunity.

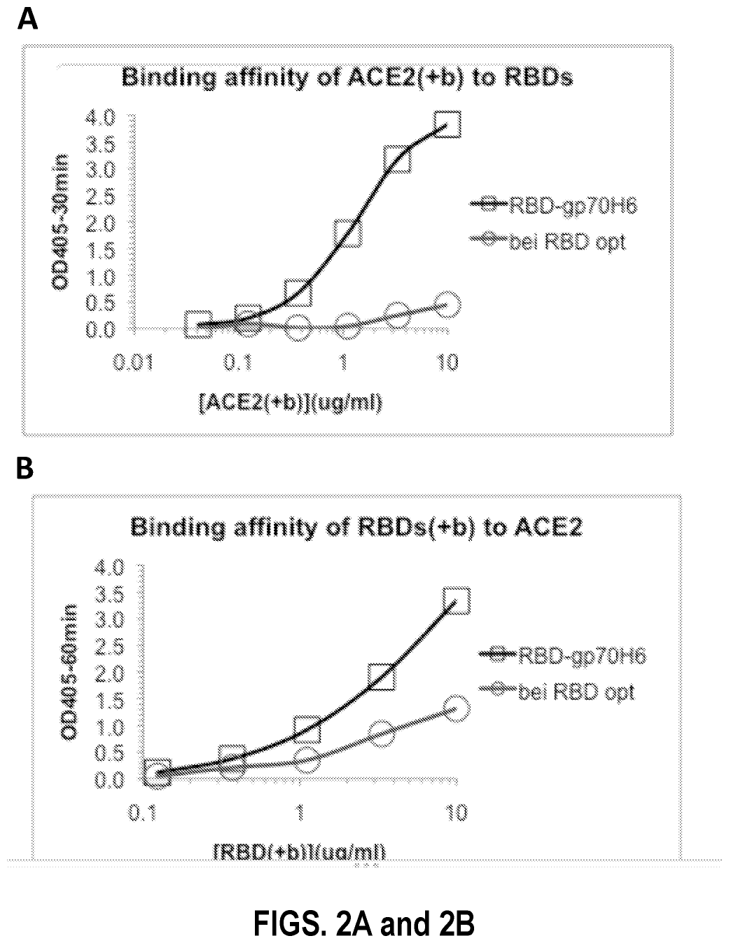

To characterize the interaction between the RBD antigen and ACE2, two procedures were examined to measure RBD/sol-ACE2 binding (FIGS. 2A and 2B). First, the gp70-RBD and RBD-BEI (or bei RBD) were directly attached to the well of an ELISA plate, and the ability of biotin-labeled soluble RBD to bind to the plate was measured (FIGS. 2A and 2B). In this format, the gp70-RBD was efficiently recognized by soluble ACE2, with an OD405 approaching 4 (saturation) at 10 μg/ml of ACE2, while the RBD-BEI antigen gave an OD of <0.5 (FIG. 2B), a level achieved with ˜0.2 μg/ml of the gp70-RBD form (FIG. 2A), an apparent 50-fold increase in sensitivity of the gp70-RBD fusion protein. Using the soluble ACE2 as the capture antigen resulted in a less efficient detection of binding, and in this case too, the gp70-RBD antigen was significantly more sensitive than the RBD-BEI antigen, with an approximately 5-fold increase in sensitivity for the gp7-RBD antigen.

This RBD-ACE2 binding inhibition assay, as demonstrated above, is a reasonable surrogate for the protective antibody titer of convalescent sera, particularly for characterizing neutralizing antibodies that target the same binding site on ACE2 as RBD. Indeed, in the present study, it was found that convalescent sera with high levels of anti-RBD titers were able to significantly inhibit the RBD-ACE2 interaction, suggesting that the RBD-ACE2 binding inhibition assay is useful for measuring the functional activity of this antibody population. This was further supported by a recent preprint that characterized over a thousand mAbs isolated from several CoV-2 convalescent patients that showed that although most of the antibodies bound to S1 and not RBD, only the ones that recognized RBD possessed neutralizing activity, and that only about one-third of the anti-RBD antibodies possessed neutralizing activities (Rogers, T. F., et al., Science, 2020: p. eabc7520 et al.). Furthermore, they showed that the ability to compete for binding of RBD to ACE2 correlated well with neutralizing activity, arguing that this is the dominant mechanism for neutralization of the virus. An important advantage of the binding competition assay is that it is readily amenable to a high throughput format and not more complicated than the standard binding assay.

### Example 4

### Use of SARS-CoV-2 Antigens for Profiling of Protective Antibodies

Viral infections typically induce neutralizing antibodies that protect against infection and help clear the virus. The primarily target of coronavirus neutralizing antibodies is the trimeric spike (S) glycoprotein (Temperton, N.J., et al., Emerg Infect Dis, 2005. 11(3): p. 411-6; Tripp, R. A., et al., J Virol Methods, 2005. 128(1-2): p. 21-8). The S protein contains two subunits: S1, which mediates viral attachment to the host cell, and S2, which is involved in viral and cellular membrane fusion and viral entry. Neutralizing antibodies against SARS-CoV-2 have already been reported (see, e.g., Lai, S. C., et al., J Biomed Sci, 2005. 12(5): p. 711-27). However, precedents exist for antibody responses having opposite effects in viral infections. For example, antibody responses may cause enhancement of Dengue virus infection and significantly increase the risk of severe disease. Of concern are reports that SARS immune sera can exhibit antibody-dependent infection of human macrophages in vitro and that anti-spike IgG caused severe acute lung injury in a SARS-CoV macaque model by skewing macrophage responses during acute SARS-COV infection.

The profiling of the protective antibody response will provide critical insight into the nature of antibodies needed to be induced by a successful vaccine. It is therefore important to characterize immune serum/plasma for protective effects as well as for infectivity-enhancing activities and related negative effects and determine the roles of antibody affinity and titer, epitope specificity, and antibody isotype in these effects. Such studies will help determine the role of antibody responses on the clinical outcome of SARS-CoV-2 infection. They will also inform convalescent plasma screening strategies associated with the use of convalescent plasma for treating COVID-19.

To this end, serum samples from three groups of SARS-CoV-2 infected individuals presenting with no, mild to moderate, and severe symptoms are obtained (see Table 3). Specimens are accompanied by demographic data, and clinical data (including death) are obtained through chart review. In addition, donors who have had documented COVID-19 are recruited. This group represents recovered symptomatic patients. Serum aliquots are banked and used for the study.

To describe antibody profiles among three groups of SARS2-CoV-2 infected individuals and determine which profiles are associated with, e.g., (1) individuals currently being asymptomatic, (2) those currently presenting with mild or moderate symptoms, (3) those that currently have severe/very severe symptoms, and (4) convalescent individuals.

TABLE 3
Patent Profiles

| | | | IRB | |
|---|---|---|---|---|
| Clinical | | Study | protocol | |
| presentation | Population | design | status | Status |
| Asymptomatic | Healthcare | Longitu- | Approved | Ongoing - >800 |
| | workers | dinal | | baseline |
| | | | | specimens |
| | | | | already collected |
| Mild - | COVID-19 | 3-arm | Approved | Ongoing -- |
| Moderate | out- and | clinical | | |
| | in-patients | trial | | |
| Severe - Very | COVID-19 | Conve- | Submitted | Not Started |
| Severe | in-patients | nience | | |
| | Non-ICU | samples | | |
| | and ICU | | | |
| Convalescent | Recovered | Conve- | Approved | Enrolment starts |
| | COVID-19 | nience | | on Apr. 14, 2020 |
| | patients | samples | | |

### Serum Antibody Assays

The assay format is a conventional ELISA utilizing antigen-coated polystyrene 96-well microtiter plates in which antigen-bound serum antibodies are detected by the colorimetric signal generated by a mixture of appropriately conjugated secondary antibody (e.g., anti-IgA, anti-IgM, anti-IgG). Viral antigens are the gp70-RBD fusion protein and additional recombinant viral antigens (full-length S1, full-length S2 or fragments thereof, nucleocapsid) produced in house in Expi-293 cells or obtained commercially. Positive control sera were obtained from COVID-19 patients. Negative control sera fr are obtained from SARS-CoV-2 PCR-negative (uninfected) subjects. Plasma or serum is heat-inactivated at 56° C. or 60° C. for 60 min prior to testing (Yunoki, M., et al., Vox Sang, 2004. 87(4): p. 302-3; Rabenau, H. F., et al., Med Microbiol Immunol, 2005. 194(1-2): p. 1-6.). The cut-off value was determined on the basis of values obtained with >700 negative control sera.

In addition to targets, titers, and isotypes, functional assays are also performed. For example, a high-throughput ELISA-based assay is performed, which determines whether test serum antibodies interfere with binding of host ACE2 receptor to the RBD of the S1 subunit immobilized to the solid phase by a capture antibody (also see EXAMPLE 3). Also, a conventional neutralization assay in which test serum antibodies are added to Vero cells (epithelial cell line) infected with luc-expressing lentiviral-based pseudovirus complemented with SARS-CoV-2 spike protein is performed to determine the effect of serum addition on luciferase activity. In addition, a neutralization assay is performed with virulent SARS-CoV-2 (untagged or tagged with fluorescent labels such as GFP) in the BSL3 laboratories, using standard plaque assays or fluorescence read-outs to determine infection.

### Results

Strain-specific antibody responses are expected when using the spike protein as solid-phase antigen, since low homology (<40%) exists between the spike proteins of members of different CoV subgroups. In contrast, nucleocapsid proteins are more conserved among coronaviruses and can potentially induce cross-reactive antibody responses. Antibody titers are expected to track with viral load, as antibody responses are usually sensitive to antigen levels. We may also find that favorable outcomes are accompanied by high titers of neutralizing antibodies. In addition, it will be determined whether serum IgG and/or secretory IgA are associated with protective responses. In the event that the protective responses are associated with secretory IgA, antibodies in saliva will also be analyzed, which may be responsible for local protective immune responses.

### Example 5

### Venue-Based Approaches to Seroprevalence Studies of SARS-CoV-2 Infection in New Jersey

Studies of the seroprevalence of SARS-CoV-2 infection are critically important. First and foremost, calculating the risk of disease and death caused by this novel infection requires reliable estimates of the number of infected individuals. To date, the knowledge about indexes of COVID-19 morbidity and mortality is shaped by local or national testing policies. The total number of infected individuals, which is currently unknown, is best attained by detecting circulating antibodies against viral proteins.

Seroprevalence studies must be local. The epidemiology of SARS-CoV-2 infection likely varies from location to location for both biological and social reasons. Age, gender, and ethnicity will very likely affect susceptibility to infection and disease, due to their effect, for example, on activity and expression levels of the host receptor for this virus (ACE2). Cultural behaviors and socioeconomic status will also affect spread and host susceptibility in multiple ways. Thus, every health jurisdiction needs information about its COVID-19 epidemic in order to combat its spread.

Essex and Hudson counties in New Jersey were selected for this study, because they are densely populated (6,000 and 14,415 residents per square mile) and diverse (largely African American and Latinx; 61.2% and 54%). Moreover, these counties have large numbers of people living in poverty (16.7% and 17.1%), with moderate income inequality, and over 12% uninsured. Additionally, the area has a confluence of transportation modes, including local and regional trains, commuter lines in and out of New York City, and an international airport. Thus, it is likely that large numbers of residents may become infected with SARS-CoV2 and that a large proportion of them may not have the ability to take precautionary measures (e.g., social distancing, self-quarantine) or adequate access to testing and medical care.

Knowing the total number of infected individuals can help monitor the epidemic, model the current status of immunological protection in the population, provide information on the effectiveness of prevention and control measures, and design rational interventions against the circulation of the SARS-CoV-2 virus moving forward. To determine population prevalence of SARS-CoV-2, quasi-probability-based sampling of the general population via a venue-based approach is conducted.

### Population Sero-Surveillance

General population health surveys requiring access to clinical specimens cannot follow designs in which study subjects are not encountered in person (e.g., as in telephone surveys) or are encountered in environments, such as households, that require intensive resources in relation to specimen collection. A venue-based approach is conducted to reach the general population for testing for SARS-CoV-2 antibodies while collecting symptom and underlying condition data from a diverse sample of the population. Recruitment for this cross-sectional survey and cohort follows the methodology of the venue-based approach as used by the CDC for National HIV Behavioral. As a universe of venue-day-time (VDT) periods where the population can be found, grocery stores are used as a sampling frame. A simple random sample of VDT is drawn with equal probability of being sampled is used to select venues-day-times to conduct study events. The considerations for choosing grocery stores focus on accessibility and the fact that a large majority of adults, often with children, regularly access these venues frequently. Therefore, grocery stores are highly suitable for recruiting subjects across various age groups. Moreover, the informed selection of stores targets subjects of various ethnicities and socioeconomic statuses. At the randomly sampled VDT, subjects are intercepted crossing a pre-designated line or zone, greeted, assessed for eligibility, and recruited.

The procedures for VBS recruitment are: (i) drawing a simple random sample of 14 VDT to fill a calendar of recruitment events each month; (ii) conducting enumeration: systematic enumeration by clicker counting of all persons entering a predetermined zone at the randomly selected VDT over the four-hour period; and (iii) intercepting enumerated subjects by staff to determine eligibility, invite participation, conduct informed consent, and enroll (intercepts continue until all outreach staff are occupied, and resumes as soon as one staff member is again available; thus recruitment is consecutive at the VDT according to staff availability with no selection by the field team); (iv) participants complete a self-administered, computer-assisted questionnaire in a nearby private location; and (v) blood drawing for SARS-CoV-2 antibody testing.

One thousand one hundred fifty (1150) persons are sampled to determine SARS-CoV-2 prevalence of 0.5-1.0% with a 0.5% margin of error at a 95% confidence level. It is anticipated that 57.5 sampling events are completed over a three-month period to achieve our sample size. Sampling promptly is started after compiling the list of grocery stores in the study counties. In addition, sampling is repeated after 6 and 12 months to enable an analysis of potential trends.

The ELISA, as described above, is used as the initial assay format for this study (see EXAMPLES 3 and 4). Given the breadth and frequency of attendance to grocery stores and the attention to stores attended by county residents of diverse socioeconomic status, the above choice of recruitment sites should be adequate. However, if it is found that grocery store attendance is biased towards women, the statistical basis is adjusted for this bias using underlying demographic data. In this study, whether any correlation exists between these two responses (e.g., seropositives to SARS-CoV-2 versus responses to the common CoV), providing evidence of cross-protection or, most likely, lack thereof. In addition, if antibodies target the receptor-binding domain of the SARS-CoV-2 spike protein, whether these antibodies are neutralizing and, therefore, protective is also determined.

### Example 6

Convalescent plasma administration has increasingly become an emergency therapeutic approach during the current COVID-19 crisis. Due to the present situation, screening of donor plasma must be speedy, and its characterization is often cursory. Moreover, the effects of plasma donation on outcome must be studied in-depth to understand how the donation affects plasma markers of inflammation and disease severity. On the donor side, antibody profiles (targets, isotypes, functions such as neutralizing or enhancing activity) are characterized. On the recipient side, rapid effects (CBC, pulmonary function, and plasma markers), as well as medium-term (anti-SARS-CoV-2 antibody titers) effects of the plasma donation, are determined. Among plasma markers, in addition to cytokines, LDH, components of the renin-angiotensin system and of the coagulation cascade are measured. In addition, an unbiased, mass spectrometry-based proteomics approach to identify additional markers of COVID-19 severity is performed.

Convalescent plasma therapy can save lives during this COVID-19 pandemic. The methods disclosed herein are useful for characterizing the antibody profiles of donors and their effects on recipients. Using the above-described ELISA and SARS-CoV-2 antigens, such as RBD of the S1 subunit of the spike protein, S1 subunit, S2 subunit, or nucleocapsid protein, the disclosed methods can detect various antibodies (e.g., IgM, IgG, IgA). Currently, more than 200 serum samples from convalescent donors have been characterized using the disclosed methods.

TABLE 4
Example EUSA plate design ^{a}

| | | Subject ID | | | | | |
|---|---|---|---|---|---|---|---|
| | | SA00865580 | | | | | |
| | | 1 | 2 | 3 | 4 | 5 | controls |
| HCW80 | A | DS0002672 | DS0002607 | DS0002656 | DS0002647 | DS0002627 | 25 |
| | | | | | | | POS |
| | B | DS0002623 | DS0002612 | DS0002657 | DS0002665 | DS0002626 | 50 |
| | | | | | | | POS |
| | C | DS0002621 | DS0002624 | DS0002664 | DS0002610 | DS0002620 | 100 |
| | | | | | | | POS |
| | D | DS0002611 | DS0002615 | DS0002653 | DS0002613 | DS0002617 | 25 |
| | | | | | | | NEG |
| | E | DS0002630 | DS0002635 | DS0002654 | DS0002608 | DS0002632 | 50 |
| | | | | | | | NEG |
| | F | DS000263 | DS0002614 | DS0002663 | DS0002622 | DS0002625 | 100 |
| | | | | | | | NEG |
| | G | DS0002609 | DS0002629 | DS0002655 | DS0002605 | DS0002628 | NO |
| | | | | | | | PRIM |
| | H | DS0002618 | DS0002634 | DS0002652 | DS0002616 | DS0002633 | NO |
| | | | | | | | ANTIG |
| | | ID barcode | | | | | |
| | | SA00865580 | | | | | |
| | | 1 | 2 | 3 | 4 | 5 | controls |
| HCW80 | A | FS04274015 | FS04272929 | FS04273345 | FS04273446 | FS04305412 | 25 |
| | | | | | | | POS |
| | B | FS04272537 | FS04272930 | FS04273351 | FS04273447 | FS04305414 | 50 |
| | | | | | | | POS |
| | C | FS04272996 | FS04272934 | FS04273252 | FS04305317 | FS04305416 | 100 |
| | | | | | | | POS |
| | D | FS04272999 | FS04272529 | FS04273254 | FS04305318 | FS04306082 | 25 |
| | | | | | | | NEG |
| | E | FS04273030 | FS04306375 | FS04273538 | FS04305219 | FS04306179 | 50 |
| | | | | | | | NEG |
| | F | FS04272866 | FS04272481 | FS04273541 | FS04305505 | FS04306181 | 100 |
| | | | | | | | NEG |
| | G | FS04272897 | FS04272513 | FS04273544 | FS04305509 | FS04306184 | NO |
| | | | | | | | PRIM |
| | H | FS04272902 | FS04272489 | FS04273443 | FS04305512 | FS04306466 | NO |
| | | | | | | | ANTIG |
| | | Subject ID | | | | | |
| | | SA00865580 | | | | | |
| | | 6 | 7 | 8 | 9 | 10 | controls |
| HCW81 | A | DS0002606 | DS0002682 | DS0002638 | DS0002661 | DS0002677 | 25 |
| | | | | | | | POS |
| | B | DS0002619 | DS0002681 | DS0002640 | DS0002648 | DS0002675 | 50 |
| | | | | | | | POS |
| | C | DS0002646 | DS0002680 | DS0002636 | DS0002671 | DS0002670 | 100 |
| | | | | | | | POS |
| | D | DS0002660 | DS0002676 | DS0002637 | DS0002659 | DS0002651 | 25 |
| | | | | | | | NEG |
| | E | DS0002684 | DS0002639 | DS0002644 | DS0002658 | DS0002650 | 50 |
| | | | | | | | NEG |
| | F | DS0002687 | DS0002641 | DS0002645 | DS0002662 | DS0002679 | 100 |
| | | | | | | | NEG |
| | G | DS0002683 | DS0002642 | DS0002673 | DS0002685 | DS0002678 | NO |
| | | | | | | | PRIM |
| | H | DS0002686 | DS0002643 | DS0002649 | DS0002674 | NJMS3 | NO |
| | | | | | | | ANTIG |
| | | Subject ID | | | | | |
| | | SA00865580 | | | | | |
| | | 6 | 7 | 8 | 9 | 10 | controls |
| HCW81 | A | FS04306467 | FS04273665 | FS04270853 | FS04270945 | FS04271495 | 25 |
| | | | | | | | POS |
| | B | FS04306468 | FS04273669 | FS04270854 | FS04270946 | FS04271496 | 50 |
| | | | | | | | POS |
| | C | FS04304642 | FS04273670 | FS04270855 | FS04270948 | FS04271522 | 100 |
| | | | | | | | POS |
| | D | FS04304645 | FS04273841 | FS04270856 | FS04270952 | FS04271527 | 25 |
| | | | | | | | NEG |
| | E | FS04274309 | FS04270849 | FS04271041 | FS04271143 | FS04271528 | 50 |
| | | | | | | | NEG |
| | F | FS04273729 | FS04270850 | FS04271044 | FS04271144 | FS04270471 | 100 |
| | | | | | | | NEG |
| | G | FS04273636 | FS04270851 | FS04271046 | FS04271432 | FS04270658 | NO |
| | | | | | | | PRIM |
| | H | FS04273637 | FS04270852 | FS04271048 | FS04271491 | NJMS3 | NO |
| | | | | | | | ANTIG |

^{a} Each test serum is tested at 1:25 dilution in duplicate (horizontally) in columns 1-10. Columns 11 and 12 include duplicates (horizontally) of one positive control serum (rows A, B, C) and one negative control serum (rows D, E, F) at 1:25, 1:50, 1:100 dilutions, no primary control (row G), and no antigen control (row H).

TABLE 5
Examples of actual ELISA plates ^{b}

| | 1 | 2 | 3 | 4 | 5 | 6 | 7 | 8 | 9 | 10 | 11 | 12 |
|---|---|---|---|---|---|---|---|---|---|---|---|---|
| A | 1.172 | 0.707 | 0.3 | 0.257 | 0.36 | 0.326 | 0.339 | 0.247 | 0.17 | 0.24 | 1.211 | 1.303 |
| B | 1.615 | 1.667 | 0.407 | 0.386 | 0.446 | 0.296 | 0.288 | 0.353 | 0.146 | 0.171 | 1.031 | 1.011 |
| C | 0.412 | 0.388 | 2.009 | 1.893 | 0.233 | 0.212 | 0.303 | 0.266 | 0.236 | 0.245 | 0.706 | 0.675 |
| D | 0.421 | 0.405 | 0.336 | 0.327 | 0.22 | 0.179 | 0.22 | 0.225 | 0.343 | 0.305 | 0.288 | 0.307 |
| E | 0.3 | 0.272 | 0.293 | 0.314 | 0.3 | 0.223 | 0.271 | 0.249 | 0.322 | 0.339 | 0.236 | 0.285 |
| F | 0.418 | 0.398 | 0.345 | 0.35 | 0.227 | 0.26 | 0.334 | 0.294 | 0.385 | 0.38 | 0.227 | 0.281 |
| G | 0.519 | 0.544 | 0.293 | 0.288 | 0.316 | 0.298 | 0.514 | 0.469 | 0.326 | 0.275 | 0.082 | 0.157 |
| H | 0.46 | 0.546 | 0.503 | 0.413 | 0.251 | 0.237 | 0.399 | 0.3 | 1.22 | 1.244 | 0.182 | 0.235 |
| A | 0.326 | 0.319 | 0.334 | 0.33 | 0.32 | 0.443 | 0.303 | 0.241 | 0.284 | 0.242 | 1.441 | 1.603 |
| B | 0.351 | 0.337 | 0.307 | 0.307 | 0.309 | 0.264 | 0.332 | 0.356 | 0.368 | 0.328 | 1.008 | 1.152 |
| C | 0.511 | 0.424 | 0.316 | 0.286 | 0.505 | 0.462 | 0.587 | 0.314 | 0.306 | 0.202 | 0.807 | 0.938 |
| D | 0.4 | 0.371 | 0.249 | 0.17 | 0.322 | 0.234 | 0.179 | 0.178 | 0.316 | 0.218 | 0.299 | 0.336 |
| E | 0.518 | 0.494 | 0.243 | 0.235 | 0.472 | 0.49 | 0.399 | 0.279 | 0.296 | 0.242 | 0.328 | 0.277 |
| F | 0.415 | 0.375 | 0.488 | 0.522 | 0.885 | 0.301 | 0.278 | 0.218 | 0.292 | 0.241 | 0.216 | 0.274 |
| G | 0.287 | 0.243 | 0.36 | 0.268 | 2.517 | 2.065 | 0.248 | 0.204 | 0.277 | 0.279 | 0.171 | 0.089 |
| H | 0.402 | 0.424 | 0.491 | 0.474 | 0.309 | 0.271 | 0.239 | 0.203 | 2.455 | 2.234 | 0.16 | 0.135 |

^{b} Conditional background is used in each well to highlight the corresponding OD405 value reading.

### Example 7

### Development of More Specific Binding Assays for Antibodies Induced by SARS-CoV-2 Infection and Vaccination.

One issue limiting the utility of serological assays for SARS-CoV-2 is the presence of non-specific antibodies. These can either be antibodies to cross-reactive targets present in the common coronaviruses that were induced by previous infections with common cold viruses or pre-existing “sticky” antibodies detected at low dilutions in many sera that bind non-specifically to the plastic used in ELISA plates or to the proteins (e.g., BSA or BLOTTO) used to block the wells. The standard method for measuring antibody binding is to detect bound antibodies with an enzyme-labeled secondary, such as AP-labeled goat anti-human H+L chains. This reagent does not discriminate between specifically captured and non-specifically bound antibodies. The concentration of these sticky antibodies is usually low in most serum, and this issue can be bypassed by diluting out the serum 20-fold or more. However, this does not work in all cases and results in the loss of some specificities.

This example describes a novel improved ELISA procedure that avoids the detection of non-specifically-bound antibodies and results in a significantly improved correlation between binding and neutralizing titers for polyclonal sera. This involved replacing the standard secondary antibody detection reagent with an antigen, such as a biotinylated antigen (e.g., biotinylated SARS-CoV-2 RBD antigen) (FIG. 3). Bivalent IgG in sera usually binds to the coated antigen with a single arm, leaving a second arm of the IgG free to interact with the biotinylated antigen. In contrast to the existing standard ELISA method, this approach detects only the RBD-specific antibodies captured by the plated antigen but not the non-specifically bound antibodies (FIG. 3). This avoids the background due to “sticky” antibodies bound to the plate and allows the serum to be tested at higher concentrations, which increases both the sensitivity and specificity of the assays.

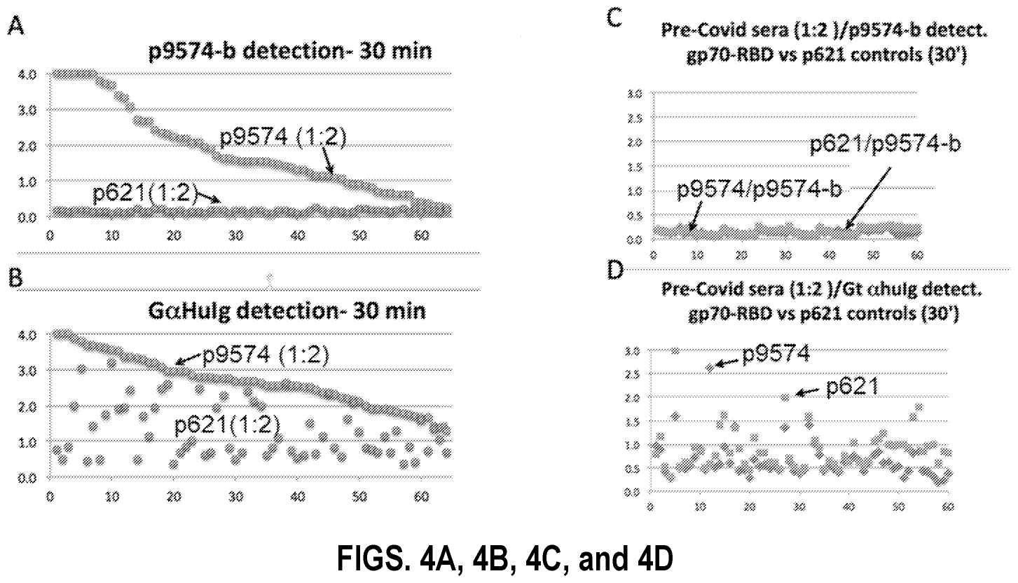

The increased specificity of the modified RBD detection assay over the standard assay is shown in FIGS. 4A-D, which compares specific binding activity against a gp70-RBD fusion antigen (p9574; diamonds) vs. the gp70 carrier domain (p621; squares) as a control for non-specific background activity. FIGS. 4A-B show patterns obtained by the disclosed method and the existing methods for an adolescent cohort of CoV-2 infected subjects, while FIGS. 4C-D show binding patterns obtained for a set of pre-COVID10 serum negative controls. The RBD/RBD assay, as disclosed, is shown in the top panels (FIGS. 4A-B), while the standard RBD/anti-human IgG assay is shown in the bottom panels (FIGS. 4C-D). All sera were tested in these assays at a dilution of 1:2.

Both methods detected binding signals for the COVID19 cohort, but while the present method gave no signals for the p621 carrier control antigen and the standard methods showed significant background activity for a large fraction of these samples (FIG. 4A-B). Similarly, while the pre-COVID sera showed no reactivity in the RBD/RBD assay against either antigen (FIG. 4C), many of these samples gave significant signals against both antigens in the standard assay (FIG. 4D). While the non-specific background of the standard assay was much lower at higher serum dilutions, they were still higher than in the new assay format. These experiments demonstrated the much greater specificity of the disclosed method using an antigen, such as an RBD antigen as the detection signal, than in the standard assay using secondary antibodies.

Improved Correlation of RBD ELISA with SARS-CoV-2 Neutralizing Activities of Convalescent Sera.

A critical question in evaluating vaccine responses is determining whether a particular antibody response relates to protection against infection and disease. Neutralization assays remain the gold standard for measuring humoral immunity, and important parameters that correlate with protection are neutralizing titer and breadth. However, neutralization assays are cumbersome and expensive to run and not ideal for mass screening studies. In fact, there are no current commercial assays for measuring neutralization titers. Therefore, there is a need for a simple binding assay that strongly correlates with neutralization titers.

For SARS-CoV-2, the major neutralization target is the RBD, and one would expect that anti-RBD titers would correlate strongly with neutralization activity. Surprisingly, it was consistently seen that antibody titers measured by standard RBD ELISAs correlated relatively poorly with neutralization. This was true even for cases where absorption experiments showed that the RBD was the dominant target for neutralization antibodies. One likely contributor to this effect is the fact that most of the RBD epitopes recognized by polyclonal antibody responses induced by infection do not mediate neutralization. An example of this is described in Rogers et al. (Rogers, T. F., et al., Science, 2020: p. eabc7520), which describes the isolation of a large panel of natural RBD-specific mAbs isolated from convalescent CoV-2 patients. Only 31% of the mAbs that bound to both S and RBD possessed neutralizing activity, while only 5% of the mAbs that bound to S but not to RBD neutralized the virus. This is particularly problematic for sera with lower antibody titers, common after mild and asymptomatic infections.

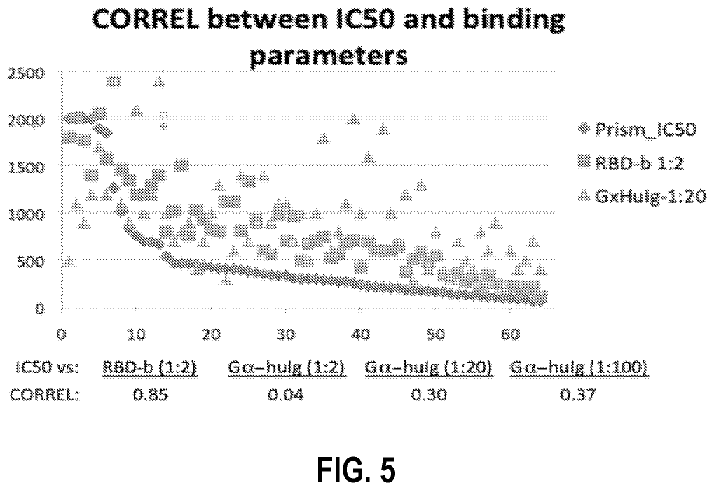

The greater specificity of the labeled antigen detection reagent resulted in a significantly improved correlation between RBD binding and serum neutralization titer (FIG. 5). The plot compares IC50s for the D614G pseudovirus (blue diamonds) with binding activity detected with the labeled RBD antigen (orange squares) vs. the standard secondary antibody assay (gray triangles) for a CoV-2-infected adolescent population. While the Correlation Coefficient values obtained with the RBD detection reagent correlated well with the IC50s (CORREL=0.85), the secondary antibody showed high non-specific reactivity at the 1:2 dilution and gave a very poor correlation coefficient (0.04). The correlation increased somewhat at higher dilutions of the primary serum (0.30 at 1:20 and 0.37 at 1:100) due to lower non-specific binding, but did not come close to that seen with the RBD detection reagent.

It was hypothesized that other factors, in addition to the reduction in non-specific binding, contribute to the improved correlation of the RBD detection method with neutralization titer. In general, this approach is detecting only a fraction of the antibodies that are detected with the standard method and may be specific for antibodies that possess higher affinities for the virus, resulting in a better correlation with functional activity. Studies with monoclonal antibodies show that while most antibodies give weaker signals in the RBD/RBD format than in the standard format, some do not react at all, and others give equivalent signals in the two assays. This indicates that epitope specificity may also be playing a role in the different reactivities in these two formats.

### Approaches to Improve the Functional Specificity of RBD-Based Binding Assays.

Although the use of RBD as the detection assay improved the correlation between binding and neutralization of polyclonal sera, this assay still allows binding of antibodies against non-functional sites in RBD, which may not contribute to neutralization. It would therefore be advantageous if those non-functional sites could be blocked, while the functional sites recognized by antibodies that block recognition of the ACE-2 receptor or that neutralize by other mechanisms be retained. One way of doing this is by mutagenizing residues in the non-functional sites that are critical for antibody binding. This requires identification of the non-functional epitopes and determination of the positions critical for antibody binding. An alternate approach that would be more efficient is blocking large regions by insertion of novel N-linked glycosylation sites (NLGS) into the non-functional domains. The steric hindrance introduced by the insertion of the relatively large N-linked glycans expressed at those positions would interfere with antibody binding to not just the modified residues but also to the surrounding residues as well.

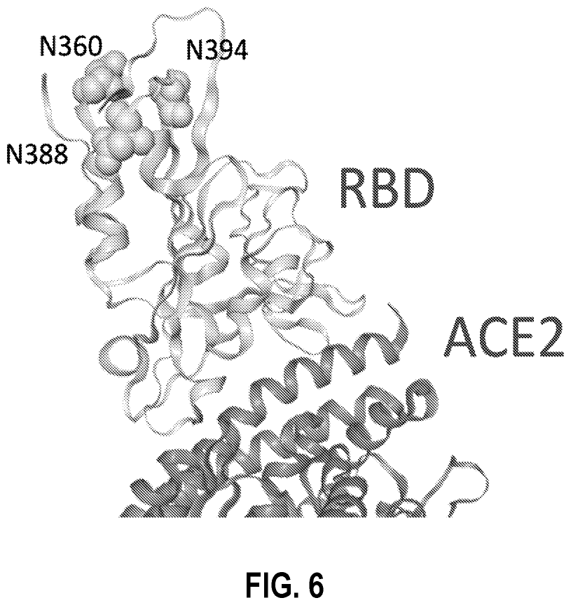

This approach requires knowledge of the effects of insertion of NLGS on RBD structure and function; substitutions that interfere with expression and correct folding and function would not be useful. N-linked glycosylation occurs at most Asparagine residues that are followed at the N+2 position by a Ser or Thr (NXS/T). An analysis has been published of the effect of converting existing Asparagine residues in RBD to NLGs by modifying the N+2 positions on the expression and ACE-2 binding, using a yeast expression system containing a library activity of a panel consisting of all possible mutations at each position in the RBD (Starr et al. Vol 182(5), 2020, 1295-1310.e20). Most (8/10) of the existing Asn residues, including the labeled Asn residues sites highlighted in yellow in FIG. 6 were shown to tolerate conversion to NLGS without negatively affecting either expression or ACE-2 binding affinities. Many of the Asn residues are adjacent to the receptor-binding motif (RBM; e.g., residues 437-508), and there was concern that NLGS inserted at one or more of these positions would interfere with function or with binding of neutralizing antibodies. The labeled Asns shown in FIG. 6 are the ones most distant from the RBD-ACE-2 interface. It was hypothesized that inserting glycans at all three of these sites would not interfere with ACE-2 binding or compete with binding of neutralizing antibodies that recognize the RBM, but would block binding of antibodies against non-functional sites that might be occluded by the new sugars.

The hyperglycosylated mutants are expressed and tested for binding to ACE-2 and recognition by panels of CoV-2 neutralizing and non-neutralizing RBD-specific mAbs. Mutants containing additional NLGs inserted into other non-neutralizing epitopes are also prepared and tested. These variants are tested for binding activity against panels of convalescent and vaccinee sera that have been carefully titered for CoV-2 neutralizing activity against viral pseudotypes containing CoV-2 S protein, using both direct RBD binding and secondary anti-Ig antibodies to quantitate binding of serum antibodies, and the correlations between binding activities in these assays and neutralization endpoints (IC₅₀s) are determined. The mutant that gives the highest correlation between levels of binding and neutralization activities are selected for screening assays that better correlate with functional activities.

1. A method of detecting an antibody or antigen-binding protein that specifically binds to a SARS-CoV-2 antigen in a sample from a subject, comprising:
contacting the sample with a SARS-CoV-2 antigen, under conditions suitable for binding the antibody or antigen-binding protein to the SARS-CoV-2 antigen; and
detecting the binding of the antibody or antigen-binding protein to the SARS-CoV-2 antigen,
wherein:
(a) the SARS-CoV-2 antigen comprises a S1 subunit or a S2 subunit of the spike protein of the SARS-CoV-2, or a fragment/variant thereof;
(b) the SARS-CoV-2 antigen comprises a receptor-binding domain (RBD) in the $1 subunit of the spike protein of the SARS-CoV-2 or fragment/variant thereof; or
(c) the SARS-CoV-2 antigen comprises an amino acid sequence of SEQ ID NO: 5 or an amino acid sequence having at least 75% sequence identity to SEQ ID NO: 5.

2. The method of claim 1, wherein the SARS-CoV-2 antigen is immobilized on a solid phase substrate either directly or through binding to a capture antibody, and wherein the method further comprises:
contacting the sample with the solid phase substrate under conditions suitable for binding the antibody or antigen-binding protein to the SARS-CoV-2 antigen, and
detecting binding of the antibody or antigen-binding protein to the solid phase substrate,
wherein the binding of the antibody or antigen-binding protein to the solid phase substrate is indicative of the subject having the antibody or antigen-binding protein that specifically binds the SARS-CoV-2 antigen.

3. The method of claim 1, further comprising identifying the antibody or antigen-binding protein after the step of detecting.

4. A method of isolating anti-SARS-CoV-2 antibodies, comprising:
contacting a sample from a subject infected with or vaccinated against SARS-CoV-2 with a SARS-CoV-2 antigen, wherein the SARS-CoV-2 antigen is immobilized on a surface of a solid phase substrate either directly or through binding to a capture antibody;
allowing SARS-CoV-2 antibodies in the sample to bind to the SARS-CoV-2 antigen;
washing the solid phase substrate;
releasing bound SARS-CoV-2 antibodies from the solid phase substrate; and
collecting the SARS-CoV-2 antibodies released from the solid phase substrate,
wherein:
(a) the SARS-CoV-2 antigen comprises a S1 subunit or a S2 subunit of the spike protein of the SARS-CoV-2, or a fragment/variant thereof;
(b) the SARS-CoV-2 antigen comprises a receptor-binding domain (RBD) in the S1 subunit of the spike protein of the SARS-CoV-2 or fragment/variant thereof; or
(c) the SARS-CoV-2 antigen comprises an amino acid sequence of SEQ ID NO: 5 or an amino acid sequence having at least 75% sequence identity to SEQ ID NO: 5.

5. The method of claim 1, wherein the step of detecting comprises detecting the antibody or antigen-binding protein bound to the SARS-CoV-2 antigen or the solid phase substrate using a second SARS-CoV-2 antigen that interacts with the antibody or antigen-binding protein, wherein:
(a) the second SARS-CoV-2 antigen comprises a S1 subunit or a S2 subunit of the spike protein of the SARS-CoV-2, or a fragment/variant thereof;
(b) the second SARS-CoV-2 antigen comprises a receptor-binding domain (RBD) in the S1 subunit of the spike protein of the SARS-CoV-2 or fragment/variant thereof; or
(c) the second SARS-CoV-2 antigen comprises an amino acid sequence of SEQ ID NO: 5 or an amino acid sequence having at least 75% sequence identity to SEQ ID NO: 5.

6. (canceled)

7. The method of claim 5, wherein the SARS-CoV-2 antigen or the second SARS-CoV-2 antigen comprises a fusion polypeptide comprising the spike protein of the SARS-CoV-2 or fragment thereof fused to a gp70 polypeptide or fragment/variant thereof, wherein:
(a) the SARS-CoV-2 antigen or the second SARS-CoV-2 antigen comprises a combination of the fusion polypeptide and the S2 subunit; or
(b) the spike protein or fragment thereof is fused to the C-terminus of the gp70 polypeptide or fragment/variant thereof.

8. The method of claim 1, wherein the spike protein or fragment/variant thereof comprises the RBD, optionally wherein:
(a) the RBD comprises the amino acids 316-542 of the spike protein; or
(b) the fusion polypeptide comprises an amino acid sequence of SEQ ID NO: 5 or an amino acid sequence having at least 90% sequence identity to SEQ ID NO: 5.

9. The method of claim 1, wherein the SARS-CoV-2 is a human or an animal SARS-CoV-2.

10. The method of claim 1, wherein the sample comprises a saliva, blood, serum, plasma, cerebrospinal fluid (CSF), peritoneal fluid, or cord blood sample.

11. The method of claim 1, wherein the subject is asymptomatic; wherein the subject either is having an active infection or has been exposed to the SARS-CoV-2; or wherein the subject has been treated with an anti-inflammatory agent or an antiviral agent or therapy.

12. The method of claim 11, wherein the antiviral agent or therapy comprises a convalescent plasma therapy.

13. The method of claim 2, wherein the solid phase substrate is selected from the group consisting of microparticles, microbeads, magnetic beads, membrane, specific monoclonal or polyclonal antibodies, and an affinity purification column.

14. The method of claim 1, wherein the step of detecting comprises detecting fluorescence or chemiluminescence or comprises a competitive binding assay, a direct ELISA or a capture ELISA.

15. The method of claim 14, wherein the competitive binding assay comprises detecting the binding of the antibody to the SARS-CoV-2 antigen in the presence of an angiotensin-converting enzyme 2 (ACE2) polypeptide or fragment thereof, and wherein the ACE2 polypeptide or fragment thereof is capable of binding to the SARS-CoV-2 antigen.

16. The method of claim 5, wherein the second SARS-CoV-2 antigen comprises a detection agent.

17. The method of claim 16, wherein the detection agent comprises a biotin moiety.

18. The method of claim 16, wherein the second SARS-CoV-2 antigen is biotinylated.

19. The method of claim 1, wherein the step of detecting comprises contacting one or more secondary antibodies with the sample, and wherein each of the one or more secondary antibodies comprises a label.

20. The method of claim 19, wherein the label is selected from the group consisting of a fluorescent label, a chemiluminescent label, a radiolabel, and an enzyme.

21. A polypeptide comprising a spike polypeptide fused to a gp70 polypeptide.

22. The polypeptide of claim 21, wherein the polypeptide comprises an amino acid sequence of SEQ ID NO: 5 or an amino acid sequence having at least 75% sequence identity to SEQ ID NO: 5.

23. The polypeptide of claim 21, wherein the polypeptide is biotinylated.

24. A polynucleotide comprising a polynucleotide sequence that encodes the polypeptide of claim 21.

25. A vector comprising the polynucleotide of claim 24.

26. A host cell comprising the vector of claim 25.

27. A kit comprising a first detection reagent comprising the polypeptide of claim 21.

28. (canceled)
